(19) United States
(12) Patent Application Publication
(10) Pub. No.: US 2026/0207121 A1
(43) Pub. Date: Jul. 23, 2026

(54) MULTI-TISSUE SEGMENTATION AND DEFORMATION FOR BREAST CANCER SURGERY

(71) Applicant: SimBioSys, Inc., Champaign, IL (US)

(72) Inventors: Joseph Ryan Peterson, Chicago, IL (US); Evandros Kaklamanos, Chicago, IL (US); Vignesh Kannan Ganesa Subramaniyan, Chicago, IL (US); John A. Cole, JR., Champaign, IL (US); Anuja Kandanatt Antony, Cary, NC (US); Amanda Parker, Oak Park, IL (US); Arda Pekis, Chicago, IL (US); Jesse Bucksot, Chicago, IL (US); Tyler M. Earnest, Steger, IL (US)

(21) Appl. No.: 19/136,095

(22) PCT Filed: Nov. 2, 2023
(86) PCT No.: PCT/US2023/078457

§ 371 Date: Jun. 5, 2025

Related U.S. Application Data
(60) Provisional application No. 63/430,645, filed on Dec. 6, 2022.

(51) Int. Cl. A61B 5/00; A61B 5/055; G01R 33/56

(52) U.S. Cl. CPC A61B 5/4312; A61B 5/0013; A61B 5/055; A61B 5/7267; G01R 33/5601; G01R 33/5608; A61B 2576/02

(57) ABSTRACT

Example embodiments may include procedures for modeling segmentation and deformation of breast tissue, useable for surgical planning and possibly other procedures. These embodiments involve applying a tumor localization, tumor segmentation, and multi-tissue segmentation procedure to 3D image data of breast tissue, then combining the results of these procedures as well as anatomical feasibilities, in determining most likely tissue types for regions of the 3D image data. Additionally or alternatively, a finite element model can be used to represent the different tissue types in the 3D image data as elements with distinct physical characteristics. Such a model can be used to simulate gravity in a posterior direction to translate the elements from the prone position to a gravity-unloaded position and then from the gravity-unloaded position to a supine position. The translated elements can be interpolated into further 3D image data with identified locations of the tissue types.

### CROSS-REFERENCE TO RELATED APPLICATION

This application claims priority to U.S. provisional patent application No. 63/430,645, filed Dec. 6, 2022, which is hereby incorporated by reference in its entirety.

### BACKGROUND

Breast cancer surgery involves a number of considerations. One of the most common is whether to perform a lumpectomy or a mastectomy. A lumpectomy removes an individual tumor from the breast, while a mastectomy removes the entire breast. The surgery that is recommended to a subject can vary based on the type of cancer, how advanced it is, and the subject's general health. For cosmetic and other reasons, many subjects prefer lumpectomies over mastectomies when the former is an option. Other considerations for surgical planning include whether the procedure is to be nipple sparing or skin sparing, the locations of incisions, the location of the tumor and margins, reconstructive surgery options, and so on. Thus, a number of factors influence the likelihood of success for breast cancer surgery, some of which are not available to medical professionals based just on traditional examination and imaging.

### SUMMARY

Magnetic resonance imaging (MRI), used in concert with computer-aided methods, can detect, diagnose, and characterize invasive breast cancers. To this end, fully automated segmentation of breast tissues can be used for quantitative breast imaging analysis, and for use in spatially resolved biophysical models of breast cancer. A suite of neural networks is presented herein that segment tumor and other tissues (e.g., chest, adipose, gland, vasculature, and/or skin), in and around the breast. The accuracy of this model was validated against the expertise of breast-specialized radiologists. With this segmentation at hand, finite element modeling (and/or other possible techniques) can be used to translate the positions of the various types of tissues from that of the prone position (in which a breast MRI is typically captured) to a supine position (in which surgical procedures are typically carried out on the subject).

Accordingly, a first example embodiment may involve obtaining 3D image data of breast tissue; applying a tumor localization procedure to the 3D image data, wherein applying the tumor localization procedure includes using a tumor localization neural network ensemble to predict 3D bounding boxes of tumor locations within the 3D image data; applying a tumor segmentation procedure to the 3D image data, wherein applying the tumor segmentation procedure includes using a tumor segmentation neural network to predict first probabilities that a first set of locations within the breast tissue contain tumor tissue; applying a multi-tissue segmentation procedure to the 3D image data, wherein applying the multi-tissue segmentation procedure includes using a tissue segmentation neural network ensemble to predict second probabilities that a second set of locations within the breast tissue contain one or more types of non-tumor tissue, and combining the second probabilities using a weighted average; and determining most likely tissue types for regions of the 3D image data based on the 3D bounding boxes, the first probabilities from the tumor segmentation procedure, the second probabilities as combined from the multi-tissue segmentation procedure, and anatomical feasibilities of the breast tissue.

A second example embodiment may involve obtaining 3D image data representing breast tissue in a prone position, wherein regions of the 3D image data are respectively labeled with predicted tissue types; transforming the regions of the 3D image data into elements of a finite element model, the elements having the predicted tissue types; based on their predicted tissue types, assigning, to the elements, respective density and stiffness parameters; and simulating, by way of the finite element model and based on the respective density and stiffness parameters, gravity in a posterior direction to translate the elements from the prone position to a supine position of the breast tissue.

A third example embodiment may involve a non-transitory computer-readable medium, having stored thereon program instructions that, upon execution by a computing system, cause the computing system to perform operations in accordance with the first and/or second example embodiment.

In a fourth example embodiment, a computing system may include at least one processor, as well as memory and program instructions. The program instructions may be stored in the memory, and upon execution by the at least one processor, cause the computing system to perform operations in accordance with the first and/or second example embodiment.

In a fifth example embodiment, a system may include various means for carrying out each of the operations of the first and/or second example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 depicts breast shape and tumor positions in the prone and supine positions, in accordance with example embodiments.

FIG. 4 depicts an overview of breast tissue segmentation and deformation modeling, in accordance with example embodiments.

FIGS. 5A, 5B, and 5C depict segmentation modeling of images of breast tissue, in accordance with example embodiments.

FIG. 6 depicts tumor localization based on DCE-MRI data, in accordance with example embodiments.

FIG. 7 depicts a neural network structure for tumor localization, in accordance with example embodiments.

FIG. 8 depicts a neural network structure for tumor segmentation inference, in accordance with example embodiments.

FIG. 9 depicts a neural network structure for multi-tissue segmentation inference, in accordance with example embodiments

FIG. 10 is a flow chart for fusion the output of tumor localization, tumor segmentation, and multi-tissue segmentation procedures, in accordance with example embodiments.

FIG. 11 is a breast tissue segmentation flow chart, in accordance with example embodiments.

FIG. 12 depict a cubic voxel that can be decomposed into tetrahedral elements, in accordance with example embodiments.

FIG. 13 is a flow chart for estimating an unloaded state of breast tissue from a prone state, in accordance with example embodiments.

FIG. 14 is a series of images depicting simulated lateral movement of breast tissue from an unloaded state to a supine state, in accordance with example embodiments.

FIG. 15 is a breast tissue deformation flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Example Computing Devices and Cloud-Based Computing Environments

The embodiments herein involve advanced image modeling, simulation, and prediction of breast tumor size, location, and/or movement when a subject is in various positions and based on subject breast characteristics. These techniques provide desirable input for consideration in order to determine the complexity and risks of a lumpectomy procedure. Further, the techniques can also be used to simulation the impact of a lumpectomy on the shape and form of a breast. As many aspects of these embodiments are computer implemented, example computing device and system embodiments are described below.

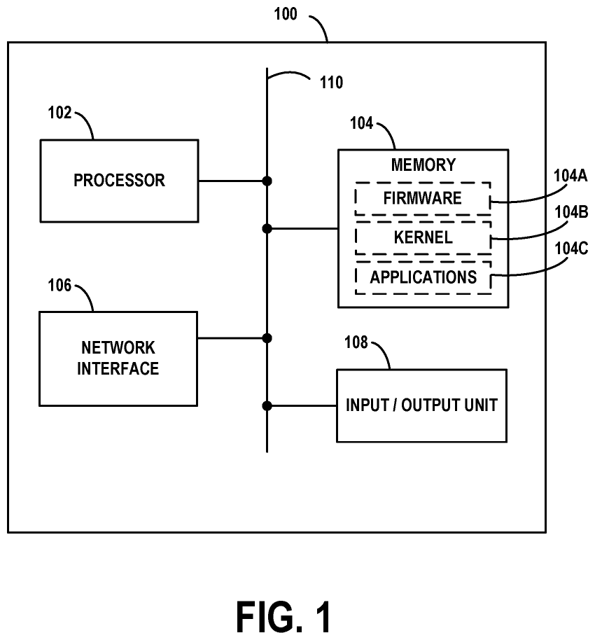

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, one or more computing devices like computing device 100 may be deployed to support the embodiments herein. The exact physical location, connectivity, and configuration of these computing devices may be unknown and/or unimportant to client devices. Accordingly, the computing devices may be referred to as “cloud-based” devices that may be housed at various remote data center locations.

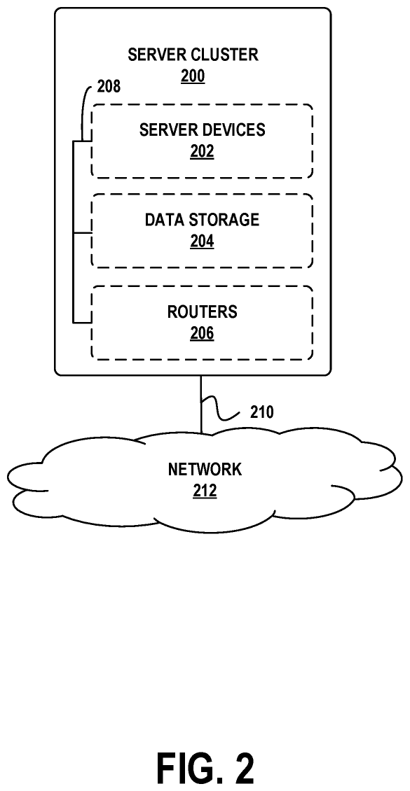

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as HTML, the eXtensible Markup Language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

### II. Surgical Options for Breast Cancer

When diagnosed with breast cancer, a subject may have several surgical options. Generally speaking, these options fall into two main treatment categories: lumpectomy and mastectomy. While both procedures aim to remove cancerous tissue from the breast, they differ in their extent of tissue removal.

Lumpectomy, also known as breast-conserving surgery or partial mastectomy, involves the removal of the tumor (herein, the term tumor is used to refer to cancerous tissue or any other malignancy) along with a small margin of healthy tissue. The primary goal of a lumpectomy is to eradicate the tumor while preserving the breast. This procedure is typically followed by radiation therapy to target any remaining cancer cells. Lumpectomy is often recommended for subjects with early-stage breast cancer where the tumor is relatively small and localized. It may also be suitable for certain cases of ductal carcinoma in situ (DCIS), a non-invasive form of breast cancer affecting the milk ducts.

Lumpectomy has the advantage that it can help preserve the natural appearance of the breast, maintaining its shape and contour. It is also a less invasive procedure than mastectomy, has a shorter recovery time, and often exhibits similar survival rates as that of mastectomy. On the other hand, there is a possibility of recurrence of the cancer if the tumor is not adequately removed and there is residual cancer in the breast. In some cases, partial reconstruction of the breast is recommended or desired after a lumpectomy.

Mastectomy involves the complete removal of the breast tissue, including the nipple and areola. Depending on the extent of the procedure, mastectomy can be classified into several types, such as total mastectomy (removal of the whole breast), radical mastectomy (removal of the whole breast, lymph nodes under the arms, and chest wall muscles under the breast), or modified radical mastectomy (similar to radical mastectomy but sparing the chest wall muscles and possibly some of the lymph nodes). Reconstruction of the breast can be done simultaneously or at a later time. Mastectomy is typically recommended for large tumors, when cancer has spread to multiple areas of the breast, or when it is in line with subject preference.

By removing the breast (and possibly surrounding tissue) in its entirety, mastectomy reduces the likelihood of recurrence and often eliminates the need for radiation therapy (which can be beneficial to subjects with certain conditions that make reception of radiation risky). However, the permanent loss of the breast can have psychological and emotional implications for some subjects. Further, mastectomy has a longer recovery time than lumpectomy. On the other hand, breast reconstruction can be an option for mastectomy subjects.

The determination of how to treat breast cancer, i.e., with lumpectomy or mastectomy, is an individualized decision between a subject and their doctor. Surgical planning relies heavily on predicting the cosmetic outcome of the surgery, which involves consideration of not only the nature and complexity of the surgery itself, but also the appearance of the post-surgery breast when the subject is in a natural position (e.g., standing or laying down). The ultimate decision may be based on a number of additional factors, such as breast size, shape, and tissue density.

Breast size influences the amount of breast tissue that needs to be removed in a lumpectomy. In larger breasts, the tumor may be relatively small in proportion, making it easier to perform a lumpectomy while preserving a significant portion of the breast. However, there may be challenges to achieving the desired cosmetic results, as reshaping and repositioning the remaining breast tissue can be more complex. Conversely, in smaller breasts, removing the tumor while maintaining an aesthetically pleasing breast contour may be comparatively easier.

Breast shape can also impact the surgical approach and the cosmetic outcome of a lumpectomy. The contours of the breast should be considered when determining the incision route to remove the tumor while preserving the shape as much as possible. In cases where the tumor is located in a challenging position within the breast, such as near the nipple or chest wall, achieving the desired cosmetic results may be more complicated requiring additional techniques or adjustments during the surgery.

Breast tissue density refers to the composition of breast tissue in terms of glandular tissue, fibrous tissue, and fatty tissue. Dense breast tissue, characterized by a higher proportion of glandular and fibrous tissue, can make it more challenging to detect and accurately assess tumors on mammograms. This can potentially affect lumpectomy procedures, as the accurate localization and complete removal of the tumor are central concerns. Higher breast tissue density can also influence the cosmetic outcome of a lumpectomy, as this breast tissue may be less pliable. This can make it more difficult to achieve a desired breast contour post-surgery.

All of these factors make accurate prediction of breast surgery outcomes to be a difficult and complicated endeavor, for at least some portion of subjects. The outcome of the operation can be influenced by the precision of these predictions. Moreover, the post-operation physical and psychological toll on the subject can be significant if these predictions are not reasonably accurate. Therefore, it is desirable for pre-surgical techniques to take these factors into account when determining the type and nature of surgical options, as well as the likelihood of post-surgical complications.

### III. Breast Surgery Planning

A standard-of-care dynamic contrast enhanced magnetic resonance imaging (DCE-MRI) for breast cancer is commonly performed with the subject lying face down (prone) on a table with breasts positioned downward through openings in the table. However, surgery is performed with the subject on their back (supine). Having to rely on the prone-positioned DCE-MRI, which is usually represented in multiple two-dimensional (2D) images, limits the ability to accurately predict surgical outcomes. Notably, gravity will pull on a breast to elongate it when prone but flatten it when supine. Further, the relative position of the tumor is likely to change in all three dimensions (e.g., vertically as well as laterally in two dimensions) as the subject is moved from the prone to supine positions. This limits the ability to accurately predict tumor location and other complicating factors based on the 2D prone-positioned DCE-MRI input.

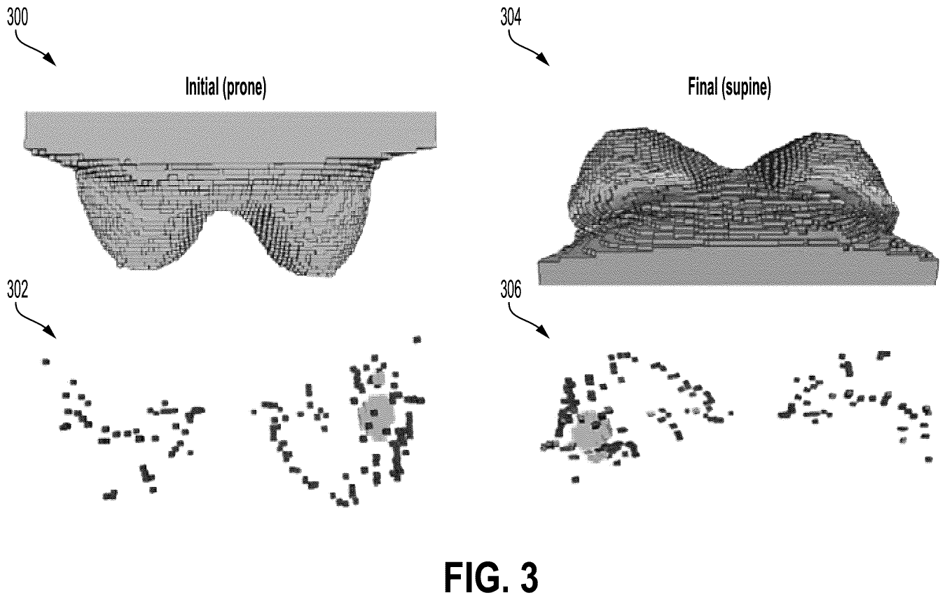

FIG. 3 depicts example breast shape and tumor positions in the prone and supine positions. Image 300 shows breasts in the prone position, as they would be arranged for MRI procedures. As noted, they are vertically elongated due to gravity. Image 302 shows the locations of various types of tissues in the prone-positioned breasts, including a tumor that is represented as a cluster of pixels to the right of the image (i.e., in the subject's left breast). Image 304 shows breasts in the supine position, as they would be arranged for surgery. Also as noted, they are vertically flattened due to gravity. Image 306 demonstrates how the tumor can move relative to its location in image 302. Notably, the tumor is closer to the chest wall and is more leftward from the subject's perspective. It is possible that the tumor has also moved in the vertical direction (e.g., closer to the subject's head or feet).

Currently, predicting these movements is challenging. A surgeon needs to project the 2D MRIs into more accurate 3D representations of breast and tumor morphologies. Subjects need to fully participate in surgical decision making and planning, but are often laypersons who are not equipped to visualize the true 3D shape and size of their breasts post-surgery. Since the type of surgery chosen (e.g., lumpectomy versus mastectomy, nipple-sparing versus skin-sparing) and its ultimate clinical and cosmetic outcomes depends not only on a surgeon's pre-operative assessment but also a subject's understanding, techniques for clear and precise 3D pre-operative visualizations in the clinic would represent a significant advance over current procedures.

### IV. Multi-Tissue Simulation and Visualization

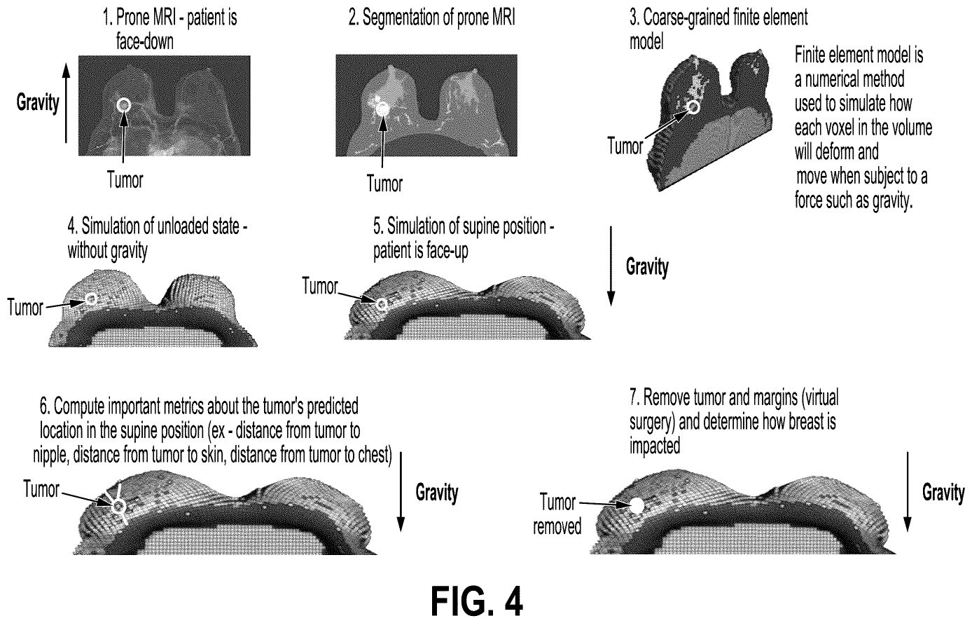

The implementations herein overcome the aforementioned limitations and drawbacks, as well as potentially other disadvantages with the previous state of the art. FIG. 4 depicts these implementations in an overview form, with further detail to follow.

At step 1 of FIG. 4, one or more DCE-MRIs of the breasts may be taken with the subject in the prone position. A tumor is shown as indicated in the left breast. To develop a 3D model of the breast, multiple DCE-MRI “slices” of the breast may be used.

At step 2, these DCE-MRIs are segmented by way of a suite of one or more convolutional neural networks (CNNs), to identify various tissues within the breasts. The output is a discrete labeling of voxels within the images with air, skin, adipose tissue, fibroglandular tissue, vasculature, tumor, and chest wall each clearly distinguished.

At step 3, a finite element model is used to simulate how each voxel will deform and move when subjected to gravity.

At step 4, this finite element model is used to simulate the breasts in the supine position without gravity loading.

At step 5, this finite element model is used to simulate the breasts in the supine position with gravity loading (i.e., deformation modeling).

At step 6, metrics related to the tumor's predicted location in the supine position are calculated (e.g., the distances from the tumor to the nipple, skin and chest wall).

At step 7, removal of the tumor and the resulting breast shape and contours are simulated to represent how the breasts will be impacted by the surgery (i.e., further deformation modeling).

Notably, the embodiments herein may not require all of these steps in the same implementation. As examples, just the segmentation modeling or the deformation modeling may be performed.

In these embodiments, tissue types can be defined as follows. Adipose tissue includes fatty tissues that make up most of the breast. Chest wall is a catch-all term for bones, muscles, and thoracic wall and cavity. Fibroglandular tissue includes the fibroglandular parenchyma of the breast. Skin is the cutaneous membrane that covers most of the body. Vasculature includes blood vessels that are large and bright enough to be visible on the DCE-MRI. Tumor is the cancerous mass(es). The right and left laterality can be considered separately in bilateral cases.

CNNs are a specialized type of artificial neural network designed for processing and analyzing structured data, particularly grid-like data such as that which is found in images. A CNN is composed of several interconnected layers that extract features from the input data and progressively learn hierarchical representations. Such a CNN may have a specific ordering of layers that facilitates feature extraction. This may include an input layer followed by one or more sets of convolutional, activation, and pooling layers. The final pooling layer may be followed by a classification layer, and the classification layer may be followed by an output layer. Like most artificial neural networks, the layers of a CNN may have sets of weights therebetween as well as layer-specific biases. The weights and biases may be applied by way of feedforward (from input layer to output layer) and updated by way of backpropagation (from output layer to input layer) procedures.

In this manner, CNNs lend themselves to many different sizes and arrangements. Example CNNs may exhibit various numbers of nodes (artificial neurons) per layer, various numbers of layers, and various arrangements of layers. For example, more complicated input data with hierarchical features may benefit from a greater number of convolutional, activation, and pooling layers. The characteristics of each type of layer is described below.

Input layer: The input to a CNN is typically two-dimensional grid-like data, such as the pixels of an image. The dimensions of the input data are represented as height, width, and depth (or channels). For example, a color image with 3 channels, one for each of red, green, and blue colors, may have a depth is 3, resulting in a total of 5 dimensions (1 for the horizontal position of a pixel, 1 for the vertical position of a pixel, and 3 for the color channel values of the pixel). Each unit of input data to the input layer may be a pixel or a block of adjacent pixels. In the case of 3D images, the input to the CNN may be a 3D grid of pixels, each with 1-3 color channels, resulting in 4-6 dimensions of data.

Convolutional layer: A convolutional layer may apply one or more learnable filters (also known as kernels or feature detectors) to the input data as received from the input layer. Each filter may perform, for example, a dot product operation between its weights and a small receptive field (patch) of the input data. This operation captures local spatial dependencies and detects visual patterns or features, such as edges, corners, or textures. Multiple filters are used to extract different types of features. The output of a convolutional layer is a set of feature maps, where each map represents the use of a specific filter across the input data.

Activation layer: An activation lay may apply an activation function to the features maps that it receives from a convolution layer. The activation function may be applied element-wise to introduce non-linearities and increase the CNN's capacity to model complex interactions between input data, identified features, and their classifications. Example activation functions used in CNNs include rectified linear unit (ReLU), sigmoid, or hyperbolic tangent (tanh). These activation functions are “smooth” in that they allow gradients to flow more easily during backpropagation, mitigating the vanishing gradient problem (where gradients become small in parts of the CNN thus hampering its ability to update weights via backpropagation), and enabling more efficient learning. Thus, gradient preservation helps the network update its parameters effectively and learn complex representations of features.

Pooling layer: A pooling layer can be used to downsample or reduce the spatial dimensions of the feature maps while preserving the information relevant to classification. Pooling helps in reducing the computational complexity of the CNN while providing translational invariance (i.e., recognizing features in the input data regardless of their location, position, or orientation). A simple form of pooling for image-related data is to apply an n×n filter with a stride of n to each n×n block of feature data from the prior activation layer. In some examples, n may be between 2 and 10.

Fully connected layer: After one or more sets of convolutional, activation, and pooling layers, the features extracted from the input data are flattened into a one-dimensional vector. This vector is then connected to a layer called a fully connected layer (or dense layer). The fully connected layer is responsible for learning the high-level representations of features and making classification predictions. It may use weight parameters to compute a weighted sum of the inputs, followed by the application of an activation function. In some embodiments, a fully connected layer could be replaced with another convolutional layer.

Output layer: The output layer of a CNN depends on the specific task at hand. For example, in image classification, it typically consists of a softmax activation function that outputs a probability distribution over different classes. In other tasks, such as object detection or semantic segmentation, the output layer may have a different structure to accommodate the specific requirements. For example, various entries in an output vector may encode for each feature identified in the input data, probabilities that the feature is one of several classes. In some examples herein, these classes may include air, skin, adipose tissue, fibroglandular tissue, vasculature, tumor, and chest wall.

CNNs can be trained using a large labeled dataset through backpropagation. This process involves comparing the network's predictions with the true labels and adjusting the weights of the network to minimize (or at least reduce) the error. This process may involve use of stochastic gradient descent (SGD) or one or more of its variants. During training, the network learns to extract relevant features from the input data, and to classify them accordingly. So-called deep CNNs refer to artificial neural networks with a large number of stacked convolutional, activation, and pooling layers. Increasing the depth of a CNN allows it to learn more complex and abstract representations. Architectural variants can be to improve CNN performance, such as residual connections (e.g., ResNet), inception modules (e.g., GoogLeNet), and attention mechanisms (e.g., Transformer-based models).

### V. Segmentation Modeling

In various implementations, a segmentation module uses quantitative signal data from a DCE-MRI machine to produce a 3D grid where each location is labeled as being one of the several clinically relevant tissues (e.g., skin, adipose tissue, fibroglandular tissue, vasculature, tumor, and chest wall), or air/background. To perform the segmentation and quantification of breast characteristics that impact surgical planning and surgery, a suite of CNNs can be employed. These segmentation CNNs were trained and validated on labeled DCE-MRI data. Such a segmentation CNN can be applied to one 2D MRI image at a time, and the classification results for each image can be combined to form a 3D model of a breast. Alternatively, the whole 3D array of image data may be segmented at once using 3D convolutions.

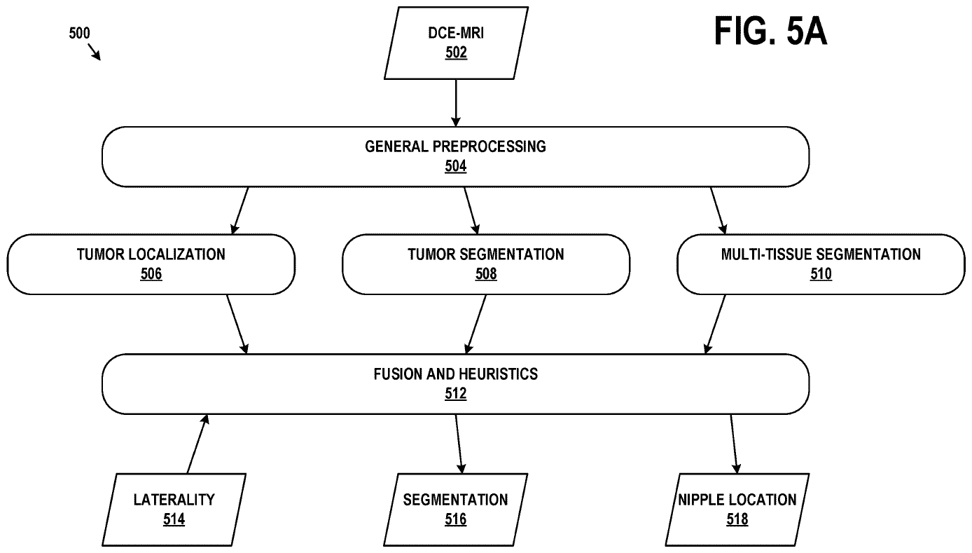

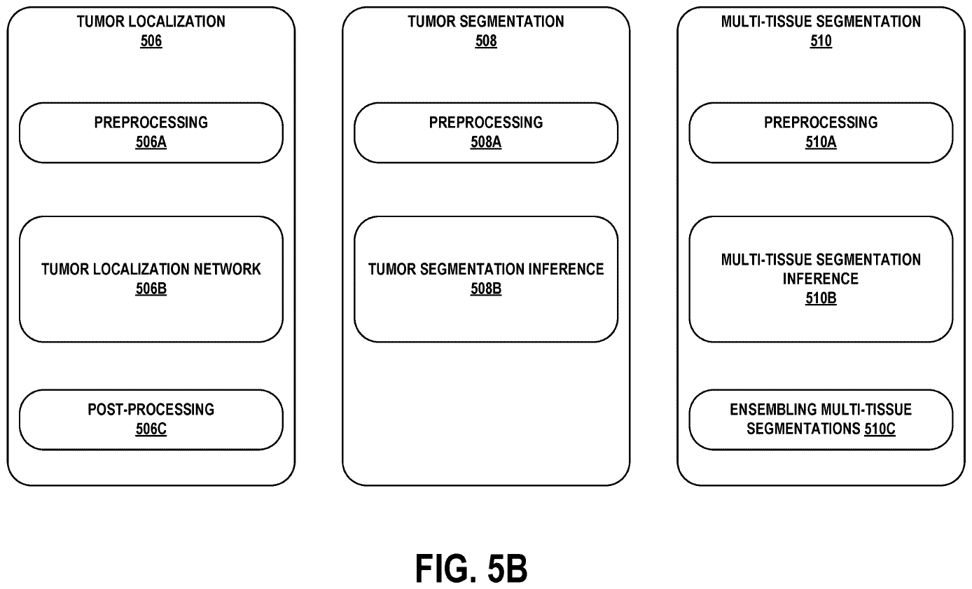

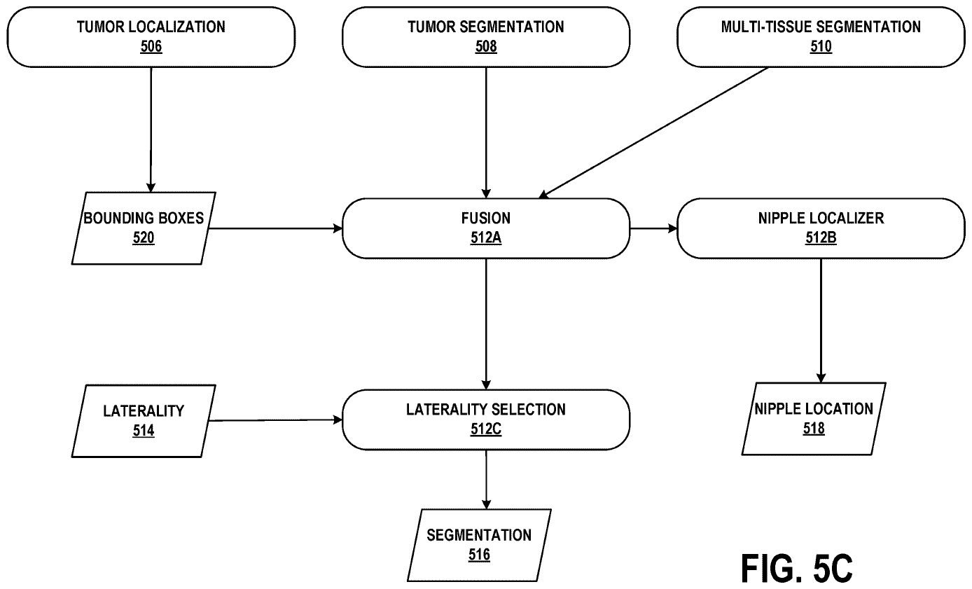

FIG. 5A depicts an overview of segmentation process 500. In this figure, as well as FIGS. 5B and 5C, quadrilaterals generally represent data and rectangles with rounded corners generally represent processing steps. Additionally, while a target population may be women diagnosed with early stage and locally advanced invasive breast cancer, other cancer stages and diagnoses may be present in the target population.

DCE-MRI data 502 may include T1-weighted (T1w), fat-suppressed images acquired with standard-of-care acquisition protocols.

General preprocessing 504 may include receiving a DCE-MRI data array, which may include a sequence of acquisition times for each frame of the DCE-MRI, in seconds, and the spacings, in millimeters, for the grid represented by the data array. General preprocessing 504 may prepare the image for downstream segmentation processing. This may include four steps: (1) temporal subsampling, (2) translational registration, (3) isotropic resampling, and (4) intensity standardization. However, more or fewer steps may be present.

Temporal subsampling may involve selecting three frames from the DCE-MRI data array. These frames may be chosen such that the relative time delays between the first and second frame, and between second and third frames, are each approximately 300 seconds (5 minutes). These delays provide to a reasonable sampling of kinetic behavior of the tissues, though other delays (longer or shorter) can be used. The first frame selected may be the first frame of the DCE-MRI data array, and the second and third frames may be selected to minimize the sum of squared deviations from the target interval. The selection of the second and third frames may be designed to minimize the error function

$${( {t}_{2} - {t}_{1} - 300 )}^{2} + {( {t}_{3} - {t}_{2} - 300 )}^{2}$$

Where t₁ is the capture time of the first frame, t₂ is the capture time of the second frame, and t₃ is the capture time of the third frame. The term 300 is based on the assumption of 300 seconds between frames. The frame times are reported by the MRI machine in the files that it provides. The selection can be done by computing all possible combinations of frame 2 and frame 3 since there usually are a limited number of frames, thus making this calculation tractable.

Translational registration may involve correcting for motion between images through alignment. For instance, the second and third frames may be registered to the first frame using phase cross-correlation. Here, phase cross-correlation can identify the relative translational shift between two similar-sized images even in the presence of noise and intensity variations. Phase cross-correlation may employ cross-correlation in Fourier space, optionally using 2D discrete Fourier transforms to achieve subpixel precision. This results in a displacement of the second and third frames by an integer multiple of the grid spacing, which maximizes the correlation of intensity across spatial sampling points.

Isotropic resampling may involve modifying the frames such that the pixel spacing is equal along all directions, thereby reducing bias. Here, the frames may be resampled to a resolution of 1 millimeter (or approximately 1 millimeter) using interpolation methods such as nearest-neighbor, linear, B-spline, and/or cubic interpolation.

Intensity standardization may involve subtracting the mean intensity from each pixel, then dividing these differences by the standard deviation of the intensity.

Once general preprocessing completes, the results thereof may be supplied to tumor localization 506, tumor segmentation 508, and multi-tissue segmentation 510. Tumor localization 506 may involve detecting the tumor using 2D object detection networks in both sagittal and axial maximum intensity projections (MIPs). Here, MIPs are used to project a maximum intensity value along a line through a 3D image into a 2D plane. This may ultimately result in the definition of one or more bounding boxes around the tumor. Tumor segmentation 508 may involve using a 3D semantic segmentation network to divide a tumor in the DCE-MRI data array into segments. Doing so may include cropping to the volume identified by the localization networks. Multi-tissue segmentation 510 may involve using an ensemble of 3D semantic segmentation networks to divide all tissues of interest (e.g., chest, adipose, gland, vasculature, and/or skin) in the DCE-MRI data array into respective segments. Each of tumor localization 506, tumor segmentation 508, and multi-tissue segmentation 510 are described in more detail below. Further, procedures for tumor localization 506, tumor segmentation 508, and multi-tissue segmentation 510 may take place independently from one another and in parallel.

Fusion and heuristics 512 may involve merging the outputs of tumor localization 506 (e.g., the bounding boxes), tumor segmentation 508, and multi-tissue segmentation 510 according to models of anatomical knowledge. This serves as a check on the localization and segmentation procedures to reduce or minimize common neural network failure modes. Fusion and heuristics 512 is also described in more detail below.

Laterality 514 may be user-specified input indicating which breast (left, right, or both) has or is expected to have a tumor. This input may be received prior to any of the processing described in FIG. 5A or after fusion and heuristics 512 completes.

Segmentation 516 is output indicating, for each voxel or 3D block of voxels in the 3D DCE-MRI data, whether the voxel is predicted to consist of or contain air, skin, adipose tissue, fibroglandular tissue, vasculature, tumor, or chest wall.

Nipple location 518 is determined based on output from fusion and heuristics 512, and is determined from the confluence of air, glandular tissue and skin for each laterality of interest.

FIG. 5B depicts tumor localization 506, tumor segmentation 508, and multi-tissue segmentation 510 in more detail. As noted, these three procedures may be performed independently from one another and in parallel.

### A. Tumor Localization

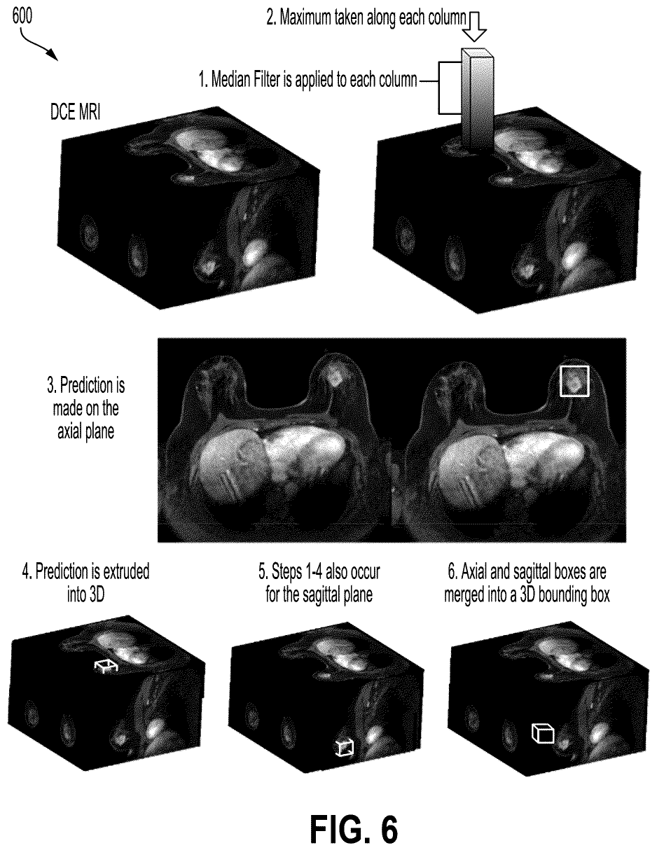

Tumor localization 506 may involve preprocessing 506A, tumor localization network application 506B, and post-processing 506C. These steps are illustrated with an example overall procedure 600 in FIG. 6. Nonetheless, some embodiments may involve more or fewer steps, or steps of a different nature.

Preprocessing 506A may involve steps (1) and (2) of FIG. 6. As noted, it is assumed that a 3D DCE-MRI image as described above is provided.

At step (1) of FIG. 6, a filter (e.g., a median filter) is applied to each column of the DCE-MRI image. A median filter, for example, can be used to reduce noise while maintaining edges in an image by sliding a window up or down each column and replacing the pixel or voxel value at the center of the window with the median value of pixels within the window. This results in filtering with respect to one axis only. Each column is processed independently in this regard. The median filter has a specific advantage in the context of breast cancer detection as skin is usually about 4-5 millimeters thick, so a median filter of 10 or so millimeters effectively ignores the skin when localizing the tumor.

At step (2) of FIG. 6, the maximum value along each column may be determined. The result is a 2D image of these maximums, as the columns have been collapsed.

Tumor localization network application 506B may involve steps (3) and (4) of FIG. 6. Notably, a localization technique may be used to predict the location of the tumor in the 2D image. A neural-network-based localization and/or object detection procedure may be used. This produces a bounding box around the predicted tumor, as shown on the right side of the image associated with step (3).

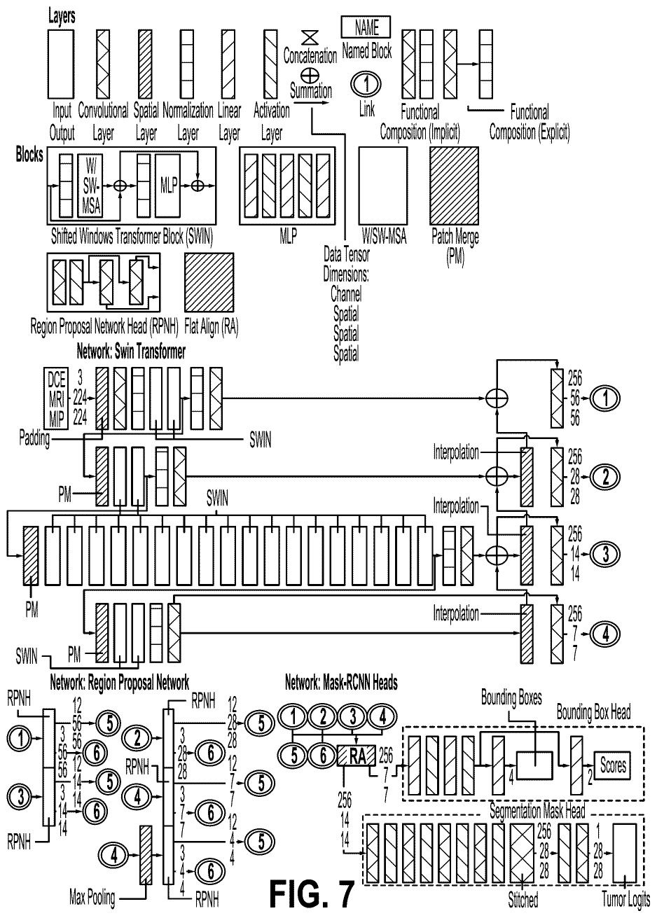

The tumor localization may be performed by way of a mask R-CNN with a Swin transformer backbone. An example of the structure of such a neural network and its processing is shown in FIG. 7.

Two tumor localization networks may be used with identical architecture and hyperparameters, but distinct learned parameters. The networks differ in the plane on which they were trained and operate. The input to each network is the respective maximum intensity projection in the plane on which each network was trained. The tumor localization networks output 2D bounding boxes for the tumor with associated confidence scores for the axial and sagittal planes.

In examples, the activation function used in the Swin transformer is Gaussian error linear unit (GELU) and the activation function used in the Mask R-CNN heads and region proposal network are rectified linear unit (ReLU). Dropout and DropPath layers are also embedded in this model, but are omitted here as they are inoperative at inference time. Bounding box proposals and outputs are limited to no more than 1000, and are de-duplicated using non-maximum suppression.

Step (4) of FIG. 6 may involve projecting this bounding box back into 3D space. Thus, step (4) may involve mapping the bounding box back into a column of the 3D image by way of extrusion. This may include extending the pixel values in the 2D image along a third axis (in this case the columnar axis) to a degree proportional to their respective intensity values. Brighter pixels are extruded more than darker pixels, creating a 3D surface.

Step (5) of FIG. 6 may involve repeating steps (1)-(4) for the sagittal plane. Thus, horizontal rows of the 3D DCE-MRI image may be subject to analogous median filtering, maximum value determination, localization, and projection as described above. Step (5) can be considered part of both preprocessing 506A and tumor localization network application 506B.

Post-processing 506C may involve step (6) of FIG. 6. The main goal of this step is merging the axial (columnar) and sagittal (row-wise) bounding boxes into a 3D bounding box. This may involve the determining the intersection of the two 2D bounding boxes. Where there is an intersection, the respective pixel values of the bounding boxes may be averaged. If there is no intersection (e.g., due to the tumor not being detected in either the axial or sagittal directions, just one of the extruded 2D boxes (e.g., the one in which the tumor was detected) may be kept and used in the overall procedure.

Notably, tumor localization network application 506B produces a set of bounding boxes and associated confidence scores in the axial and sagittal planes. Post-processing 506C proceeds independently per laterality. Here, the term “laterality” refers to which one of a paired organ is being considered. In the case of breast cancer, laterality considers the left and right breast separately.

The axial bounding boxes are grouped into left and right lateralities depending on where their centroid falls relative to the left-right median plane, and the sagittal bounding boxes are inferred separately for each half-space. The merger of the axial and sagittal bounding boxes proceeds based on the following cases: (1) there is an intersection of an extruded sagittal box and extruded axial box, (2) there is an axial box but no sagittal box, (3) there is a sagittal box but no axial box, and (4) there are no boxes predicted.

Case (1) assumes there is an intersecting extruded sagittal box and extruded axial box. Extrusion implies a third dimension is added to the box in the perpendicular axis to the originating plane. For sagittal boxes, extrusions stop at the median left-right plane of the image. If there is more than one sagittal box intersecting the axial box, the sagittal boxes are fused by taking the weighted sum of the bounding boxes relative to the confidence scores of the boxes. Given that this produces exactly one sagittal box, the axial and sagittal boxes are combined by taking their parameters in the left-right and superior-inferior axes respectively, while the shared anterior-posterior axis is averaged.

Case (2) assumes there is an axial box but no intersecting sagittal box. In this case, the extruded axial box is taken as the result.

Case (3) assumes there is a sagittal box, but no axial box. If there are multiple sagittal boxes, they are fused in the same manner as in the first case. Given that this produces exactly one sagittal box, the extruded sagittal box is taken as the result, where extrusion proceeds in the same manner as in case (1).

In case (4), there are no boxes predicted. In this case, the entire half-volume for that laterality is considered for tumor segmentations.

These procedures are advantageous because an existing 2D localization neural network can be used to perform 3D localization. Thus, a 3D localization neural network (which would be significantly more complicated than its 2D counterpart) is not required.

### B. Tumor Segmentation

Tumor segmentation 508 may involve preprocessing 508A and tumor segmentation inference 508B. Nonetheless, some embodiments may involve more or fewer steps, or steps of a different nature.

Preprocessing 508A may involve dividing the DCE-MRI image from general preprocessing 504 into 3D windows (e.g., 96 millimeters by 128 millimeters by 128 millimeters, or similar dimensions), the size of which was chosen to fit the hardware constraints (e.g., processor word size and/or bus width) of the GPU used for training. These windows can overlap by 15% of their width on each edge and cover the entire image volume. The overlap is chosen as a compromise between providing enough overlap to avoid artifacts at the edge of the window, and computational efficiency. The output of this model consists of two channels which represent positive and negative logits for the probability of tumor at each 3D window.

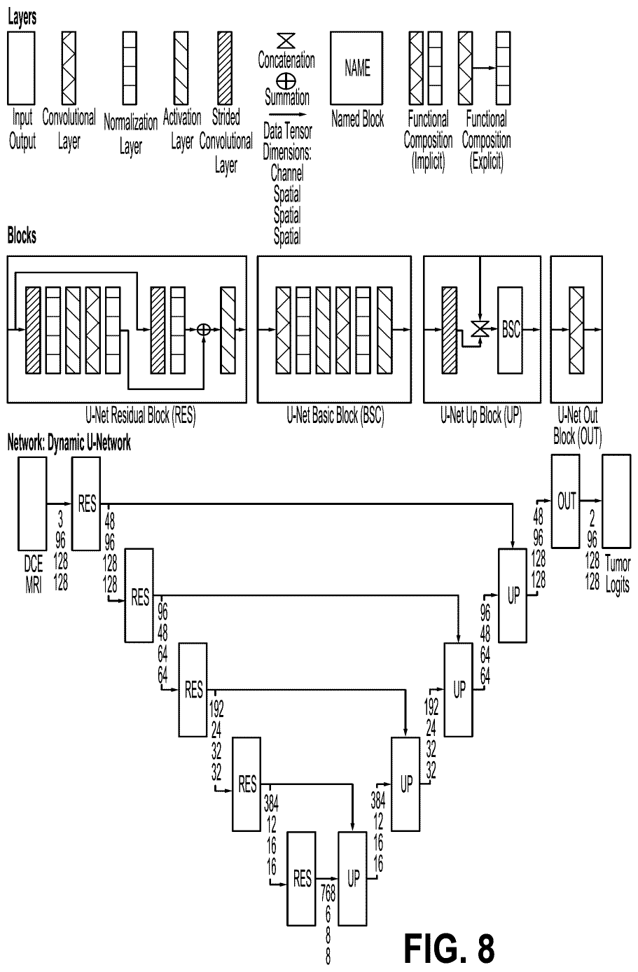

Tumor segmentation inference 508B may involve applying a neural network structure, such as the neural network of FIG. 8, to these 3D windows. The training data of this model may include a set of ground truth tumor segmentations (e.g., annotated by experts). For training purposes, rather than using sliding windows over the entire image, random windows may be sampled from each DCE-MRI of the training data such that there was a 90% chance of the window being centered on a tumor voxel and 10% otherwise. The images were augmented by randomly sampling from the following image augmentations: additive Gaussian noise, spatially correlated multiplicative Gaussian noise, rotations, scaling, elastic deformations, and drift.

The loss function used is a combination of cross-entropy loss and Dice Loss, with weights of 20% and 80% respectively. The Dice loss is calculated as:

$$ℒ ( p , g , m ) = 1 - \frac{1}{{N}_{R}} {\sum }_{r} \frac{2 {\sum }_{i} {p}_{ri} {g}_{ri} {m}_{i}}{{\sum }_{i} {m}_{i} {p}_{ri} + {\sum }_{i} {g}_{ri} + ϵ}$$

Where gᵣᵢ is 1 if voxel i is of class r and 0 otherwise, pgᵣᵢ is the predicted probability of voxel i of class r, mᵢ is a spatial mask, ε is set to 1 as a softening factor, and N_{R} is the number of classes. In this case, only the tumor is considered, so the number of classes is 1 and no voxels are masked.

The model is randomly initialized. The Adam optimizer is used with a learning rate of 0.00005. The batch size is 4 images per iteration and 200 epochs over the training dataset are performed. The model was evaluated using the Dice coefficient, over the tuning dataset. For tuning evaluation purposes, a single window centered on the true tumor segmentation was used.

The neural network of FIG. 8 belongs to the residual U-net family, which is conceptually derived from the U-net and residual net families. The activation function used is LeakyReLU. The normalization layer used is instance normalization. Dropout layers may be embedded in this model but are omitted here as they can be inoperative during inference.

The predictions for each 3D window are combined at the overlapped edges of each window. This combination may be computed pointwise and is a weighted average of the logits where the weight is the value of a Gaussian distribution centered in each window. The sigma parameters of the Gaussian are 0.125 times the width of each window dimension, though other values can be used.

### C. Multi-Tissue Segmentation

Multi-tissue segmentation 510 may involve preprocessing 510A, multi-tissue segmentation inference 510B, and ensembling of multi-tissue segmentations 510C. Nonetheless, some embodiments may involve more or fewer steps, or steps of a different nature.

Preprocessing 510A may involve may involve dividing the DCE-MRI image from general preprocessing 504 into 3D windows (e.g., 64 millimeters by 128 millimeters by 128 millimeters, or similar dimensions), the size of which was chosen to fit the hardware constraints (e.g., processor word size and/or bus width) of the GPU used for training. The windows may be spaced such that there are 16 millimeters of overlap between neighboring windows. The windows cover the entire image region with an additional 8 millimeters of padding. The window overlap is chosen to trade off the potential for edge artifacts versus the computational cost of inference.

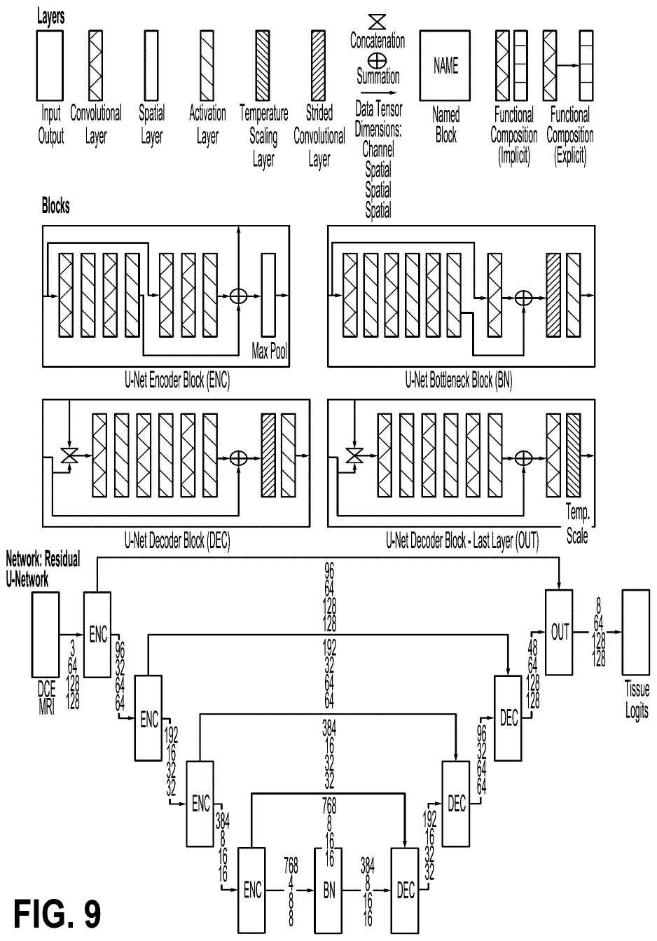

Tumor segmentation inference 510B may involve applying neural network structures, such as those of FIG. 9, to these 3D windows. Notably, five multi-tissue segmentation networks may be used (e.g., one trained to predict each of skin, adipose tissue, fibroglandular tissue, vasculature, and chest wall), each of which may have identical architecture and hyperparameters, but distinct learned parameters. The networks differ in the sample of training subjects used. The training data of this model may include a set of ground truth tissue segmentations (e.g., annotated by experts).

The training of the multi-tissue segmentation model followed a five-fold cross validation design. As such, the training dataset for each model is 80% of the combined cross-validation dataset, and the tuning dataset for each model is 20% of the combined cross-validation dataset. The tuning datasets for all five models are disjoint from each other. The training dataset and tuning dataset for each model are also disjoint from each other.

For training purposes, rather than using sliding windows over the entire image, random windows are sampled from each DCE-MRI. The images were augmented by randomly sampling from the following image augmentations: additive Gaussian noise, spatially correlated multiplicative Gaussian noise, rotations, scaling, elastic deformations, and drift.

The loss function is the sum of the cross-entropy and Dice loss functions. Dice loss is defined above. In this case, the number of classes is eight (air, skin, adipose tissue, fibroglandular tissue, vasculature, tumor, and chest wall, as well as a background class), and some voxels considered to be ambiguous are masked from both the Dice loss and cross-entropy losses.

The models may be randomly initialized. In various embodiments, the Adam optimizer may be used with stochastic gradient descent and a learning rate of 0.0001, a Nesterov momentum factor of 0.9, and a weight decay of 0.00005. In these or other embodiments, each model may be trained for 60 epochs over its training split, though more or fewer epochs may be used.

The models were evaluated over their respective tuning datasets. The evaluation was limited to voxels labeled as or predicted as adipose, glandular tissue, tumor or vasculature, or within 10 mm of the former. Metrics evaluated included Dice coefficient, precision, sensitivity, specificity, and the area under the receiver operating characteristic curve (AUC ROC). Precision is the true positive count divided by the sum of all predicted positives. Sensitivity is the true positive count divided by the sum of all positives labeled in ground truth. Specificity is the true negative count divided by the sum of all negatives labeled in ground truth. These metrics are evaluated using the predicted class which has the greatest probability for each voxel. The AUC ROC is the integral of the true positive rate relative to the false positive rate. The true positive rate is the true positive count divided by the sum of all positives labeled in ground truth (same as sensitivity). The false positive rate is the false positive count divided by the sum of all negatives labeled in ground truth. This is computed at many probability thresholds for each class to form a curve.

The models' predicted probabilities are combined using a weighted geometric average. The coefficients for the average are distinct per model per class. The weight for each model per class is determined by the IoU (intersection over union) score of the model's segmentation compared to the ground truth in the cross-validation set for the model, for the class. The weights are then normalized to sum to 1 per class and zeroed for the background placeholder class.

The neural network of FIG. 9 belongs to the residual U-net family, which is conceptually derived from the U-net and residual net families. The activation function used is ReLU.

Tumor segmentation inference 510B may further involve each network being computed over each window described above in the context of preprocessing 510A. The predictions for each window may be combined at the overlapped edges of each window. This combination can be computed pointwise and as a weighted average of the logits where the weight is the value of the tapered cosine window with scale parameter set by the window size (also known as Tukey window). This procedure results in five distinct tensors of logits (multidimensional arrays holding logit values) over the entire image volume.

Ensembling multi-tissue segmentations 510C may involve combining the five logit tensors by applying a model-specific and class-specific weighted average. The weights are calculated as described above (e.g., normalized weights based on the IoU score). The result is one tensor with predicted probabilities for the eight tissue and non-tissue classes considered (background, air, skin, adipose tissue, fibroglandular tissue, vasculature, tumor, and chest wall).

### D. Fusion of Localization and Segmentation Data

Fusion and heuristics 512 takes the output of tumor localization 506, tumor segmentation 508, and multi-tissue segmentation 510, along with laterality 514, and produces segmentation 516 and nipple location 518. FIG. 5C depicts this part of the overall procedure in more detail. Notably, tumor localization produces 506 bounding boxes 520 around the predicted location(s) of the tumor. Bounding boxes 520, along with the output of tumor segmentation 508 (positive and negative logits for the probability of tumor at each 3D window) and multi-tissue segmentation 510 (predicted probabilities for the eight tissue and non-tissue classes per 3D window).

A battery of heuristic methods may be applied to bounding boxes from tumor localization 506 and the predicted probability maps from the tumor segmentation and multi-tissue segmentation networks. These methods also accommodate for the failure states of the neural networks by applying anatomical knowledge and a certain amount of common sense to eliminate inaccurate predictions, such as those that are not anatomically feasible.

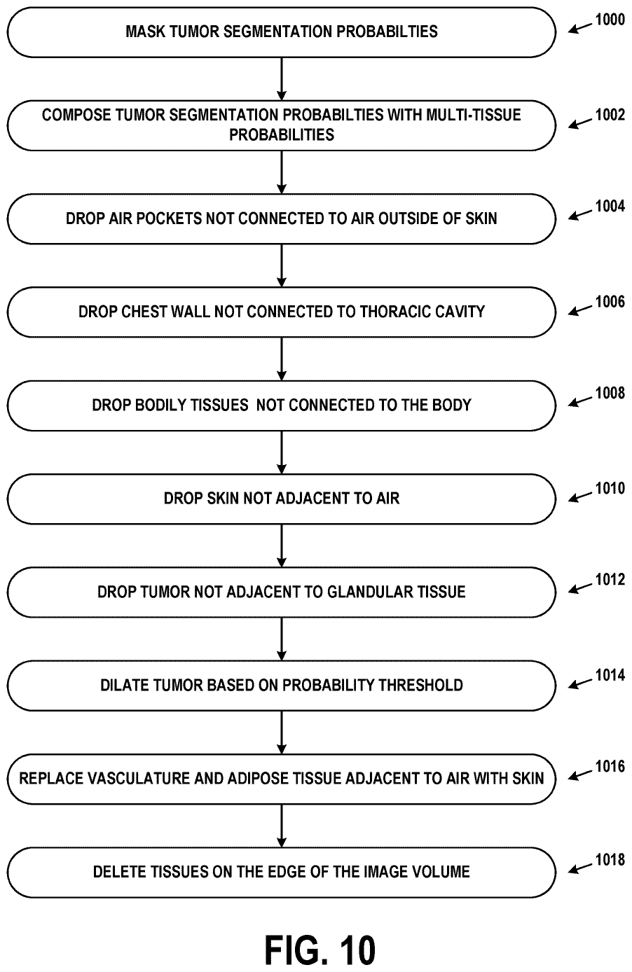

FIG. 10 depicts example steps of fusion and heuristics 512. However, more or few steps may be employed, and these steps can occur in a different order than what is shown in FIG. 10. In other words, all steps in FIG. 10 are not required and in some cases different steps not shown can be included.

At step 1000, the tumor segmentation probabilities from the tumor segmentation model are filtered according to the tumor localizer bounding boxes. This eliminates indications of tumor from most tissue outside the bounding box. A mask is constructed for tumor predictions taking a positive value where it is predicted as more likely than not that the 3D window of the DCE-MRI data is part of a tumor. This is split into connected components, which are contiguous regions of tumor predictions that are discontinuous to each other. For each connected component, the fraction of its volume that is within a tumor localization box is computed. Components that have less than 80% overlap with a tumor localization box are deleted. This mask is used to filter the tumor probabilities.

At step 1002, the filtered tumor probabilities are combined with the multi-tissue segmentation probability maps to produce an array of probabilities for each 3D window. The tumor probabilities are smoothed using a greyscale closing operation, but only where the local average probability of tumor is at least 60% (though other thresholds, such as those between 50% and 75% may be used). The multi-tissue segmentation probability map is modified by adding the tumor probability mass to glandular tissue and replacing the tumor probability with the probabilities from the tumor segmentation network tumor probability map, while suppressing the tumor probability by the vasculature probability. This overrides certain vascular predictions that are likely to be false positives. The probability map may then be renormalized to sum to 1. The highest probability for each 3D window is then used in the steps below.

At step 1004, air predictions not connected to air outside the subject are dropped by using connected component analysis. Specifically, any air component not connected to the image borders is filled in with the most common neighboring tissue.

At step 1006, chest predictions not connected to the thoracic cavity are dropped by using connected component analysis. Specifically, any chest component not connected to the largest chest component is filled in with adipose tissue.

At step 1008, tissue predictions not connected to the body are dropped by using connected component analysis. Specifically, any tissue component not connected to the largest component composed of all bodily tissues considered together is filled in with air.

At step 1010, skin predictions not adjacent to air are dropped by using connected component analysis. Specifically, any skin component that does not share at least 64 square millimeters of contact surface with air is filled in with the most common neighboring tissue (other thresholds, such as those between 36 square millimeters and 100 square millimeters, may be used).

At step 1012, tumor predictions not adjacent to glandular tissue are dropped by using connected component analysis. Specifically, any tumor component that does not share at least 64 square millimeters of contact surface with gland is filled in with the most common neighboring tissue (other thresholds, such as those between 36 square millimeters and 100 square millimeters, may be used). The rationale for this step is that breast tumors typically develop in glandular tissue.

At step 1014, the tumor is dilated subject to a probability threshold. For example, the tumor may be dilated by up to 4 millimeters, but only if the tumor probability in the region to be dilated into is at least 25% for every incremental 1 millimeter up to 4 millimeters (other thresholds, such as those between 10% and 50%, may be used).

At step 1016, vasculature or adipose tissue adjacent to air is replaced with skin. For example, vasculature and adipose tissue predictions within a 2 mm ball of air may be replaced with skin.

At step 1018, any tissues on the edge of the image volume are deleted. For example, a margin of 5 millimeters from all faces of the image volume may be replaced with the background class, representing no prediction being made for this region (other thresholds, such as those between 2 millimeters and 10 millimeters may be used).

In these embodiments, eliminating or replacing a predicted most likely tissue type may involve selecting the predicted next most likely tissue type as a replacement tissue type.

As shown in FIG. 5C, laterality 514 may be user input to the model as a laterality selection step 512C. To make this choice effective, the predicted tumor on the contralateral (non-selected) half of the image—if it exists—is replaced with a prediction of glandular tissue. If the user indicates bilateral cancer, then nothing is changed in this step.

The output of this process is segmentation 516 of the 3D DCE-MRI image data into 8 classes: background, air, skin, adipose tissue, fibroglandular tissue, vasculature, tumor, and chest wall.

### E. Nipple Localization

As shown in FIG. 5C, nipple localizer 512B may be used to determine a likely location of a nipple within the 3D DCE-MRI data. Nipple localizer 512B uses the multi-tissue probability map provided by the multi-tissue segmentation model ensemble to identify one nipple point per laterality. The output of this step is nipple location 518.

First, areas of confluence of air, glandular tissue, and skin are identified. A nipple is expected to be located on the outside of the breast, attached to the skin, and attached to milk-producing glandular tissue. Confluences are found by computing the overlap of the closing over 4 millimeters of the dilation over a radius of air probabilities, the dilation over a radius of the opening over 2 millimeters of the glandular tissue probabilities, and the closing over 4 millimeters of the dilation over a radius of the skin probabilities, where the dilation radius is increased from 1 millimeter to 8 millimeters until an overlap is identified with a confidence of at least 40%. Note that other threshold values may be used for any of these variables.

Then the overlap region is expanded using hysteresis thresholding where the strong threshold is 40% and the weak threshold is 20%. Here, hysteresis thresholding may involve, in a first step, identifying pixels or voxels with intensity values higher than the strong threshold as edges and pixels or voxels with intensity values lower than the weak threshold as non-edges. For pixels or voxels with intensity values between the strong and weak thresholds, they are identified as edges only if they are connected to pixels or voxels that are identified as edges in the first step. This connectivity analysis helps to preserve the continuity of edge lines and discard isolated pixels or voxels values caused by noise. Notably, different strong and weak thresholds may be used.

The center of mass of this region is computed and adjusted towards the posterior by 6 millimeters (or a value between 4 and 8 millimeters) to compensate for the expected thickness of the nipple-areolar complex. This computation is performed independently for the left and right halves of the image to provide predictions of the left and right nipple locations.

### F. Example Operations

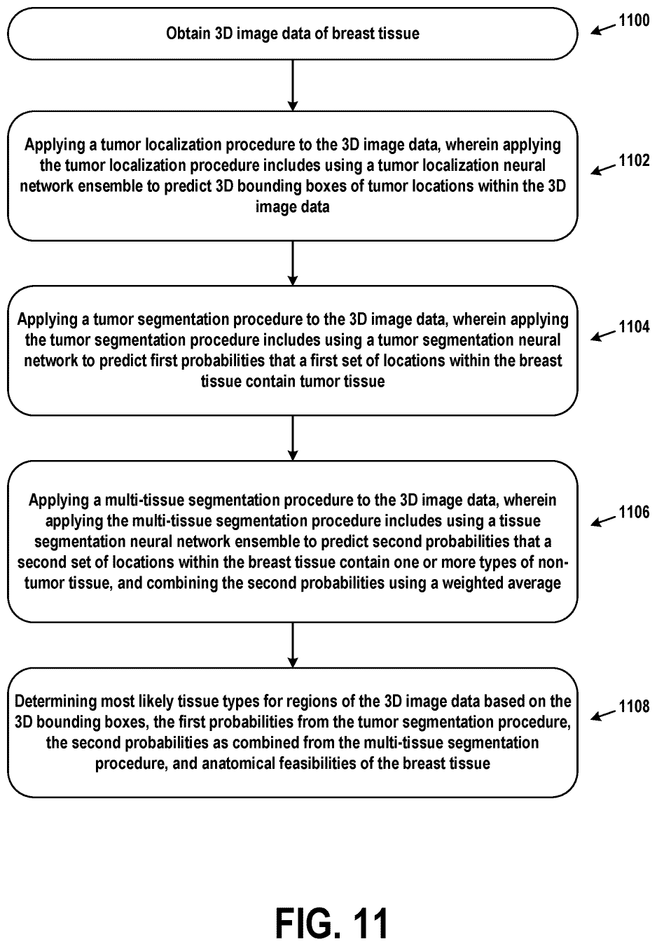

FIG. 11 is a flow chart illustrating an example embodiment. The process illustrated by FIG. 11 may be carried out by a computing device, such as computing device 100, and/or a cluster of computing devices, such as server cluster 200. However, the process can be carried out by other types of devices or device subsystems. For example, the process could be carried out by a portable computer, such as a laptop or a tablet device.

The embodiments of FIG. 11 may be simplified by the removal of any one or more of the features shown therein. Further, these embodiments may be combined with features, aspects, and/or implementations of any of the previous figures or otherwise described herein.

Block 1100 may involve obtaining 3D image data of breast tissue.

Block 102 may involve applying a tumor localization procedure to the 3D image data, wherein applying the tumor localization procedure includes using a tumor localization neural network ensemble to predict 3D bounding boxes of tumor locations within the 3D image data.

Block 1104 may involve applying a tumor segmentation procedure to the 3D image data, wherein applying the tumor segmentation procedure includes: using a tumor segmentation neural network to predict first probabilities that a first set of locations within the breast tissue contain tumor tissue.

Block 1106 may involve applying a multi-tissue segmentation procedure to the 3D image data, wherein applying the multi-tissue segmentation procedure includes using a tissue segmentation neural network ensemble to predict second probabilities that a second set of locations within the breast tissue contain one or more types of non-tumor tissue, and combining the second probabilities using a weighted average.

Block 1108 may involve determining most likely tissue types for regions of the 3D image data based on the 3D bounding boxes, the first probabilities from the tumor segmentation procedure, the second probabilities as combined from the multi-tissue segmentation procedure, and anatomical feasibilities of the breast tissue.

In some implementations, using the tumor localization neural network ensemble to predict the 3D bounding boxes of tumor locations within the 3D image data comprises: flattening the 3D image data into 2D image data for two planes of the 3D image data; using the tumor localization neural network ensemble to predict 2D tumor locations within the 2D image data; and merging the 2D tumor locations into the 3D bounding boxes.

In some implementations, merging the 2D tumor locations into 3D bounding boxes comprises determining intersections of the 2D tumor locations across the two planes.

In some implementations, determining most likely tissue types for regions of the 3D image data comprises one or more of: eliminating or replacing predicted most likely tissue types of tumor located outside of the 3D bounding boxes, or eliminating or replacing anatomically impossible predicted most likely tissue types.

In some implementations, applying the tumor segmentation procedure comprises dividing the 3D image data into 3D windows, wherein the first set of locations comprises the 3D windows.

In some implementations, applying the multi-tissue segmentation procedure comprises dividing the 3D image data into further 3D windows, wherein the second set of locations comprises the further 3D windows.

In some implementations, the 3D image data comprises two or more DCE-MRI images from different post-contrast injection time points or from two or more different MRI modalities (e.g., T1 weighted MRI, diffusion weighted MRI, T2 weighted MRI). Some segmentation networks use T1 weighted MRI data at a pre-contrast injection time point and two post-contrast injection time points, with or without multiple MRI modalities.

Some implementations may further involve: aligning the two or more DCE-MRI images; modifying the two or more DCE-MRI images to equalize pixel spacing in each dimension; and standardizing intensities of pixels within the two or more DCE-MRI images.

In some implementations, the two planes of the 3D image data are selected from axial, sagittal, or coronal planes.

In some implementations, the tumor localization neural network ensemble comprises two neural networks that were respectively trained on labeled maximum intensity projections of tumor locations within the two planes.

In some implementations, the tumor segmentation neural network was trained on randomly-selected labeled locations within 3D training images of breast tissue, wherein the randomly-selected labeled locations are biased toward tumor locations over non-tumor locations.

In some implementations, the tissue segmentation neural network ensemble comprises a plurality of tissue segmentation neural networks, one for each of a plurality of non-tumor tissue types, wherein each of the plurality of tissue segmentation neural networks was trained on labeled locations within 3D training images of their respective non-tumor tissue types.

In some implementations, the plurality of tissue segmentation neural networks include one for each of skin, adipose tissue, fibroglandular tissue, vasculature, and chest wall.

In some implementations, tissue segmentation neural network ensemble also predicts probabilities that each of the first set of locations contain air, the method further comprising determining a nipple location on a breast represented in 3D image data based on a confluence of physically adjacent or overlapping predictions of air, glandular tissue, and skin within the 3D image data.

In some implementations, the 3D image data represents the breast tissue in a prone position. These implementations may further involve: transforming the regions of the 3D image data into elements of a finite element model, the elements having their respective most likely tissue types; based on their respective most likely tissue types, assigning, to the elements, respective density and stiffness parameters; simulating, by way of the finite element model and based on the respective density and stiffness parameters, gravity in a posterior direction to translate the elements from the prone position to a gravity-unloaded position of the breast tissue; simulating, by way of the finite element model, gravity in the posterior direction to translate the elements from the gravity-unloaded position to a supine position of the breast tissue; and interpolating the elements in the supine position into further 3D image data with identified locations of the respective most likely tissue type.

### VI. Deformation Modeling

The deformation simulation described herein aids in planning the surgical removal of breast tumors by providing a surgeon with configuration estimates of subject breasts in the supine position and in the standing position, before and after surgery. The simulation takes 3D DCE-MRI image data of breast tissue as input in which the subject is in the prone position. As described above, a labeling of segments (voxels) of the breast tissues is automatically produced, and predicted most likely tissue types (e.g., tumor, skin, adipose tissue, fibroglandular tissue, vasculature, or chest wall) as well as background and air types are assigned to these segments. Alternatively, a manually-labeled map of the DCE-MRI image data may be used.

Using a finite element model, simulated external (e.g., gravitational) forces are applied to the segments in order to reflect those a subject would experience when moving from a prone position to another position (e.g., supine). A simulation is then run in which the final position and shape of the breast is iteratively solved for and this position data is saved. A 3D visualization of this final position is produced and displayed for the user. The user can interact with this display via a user interface.

### A. Optional Initialization

The following optional steps may be employed for processing the output of the segmentation modeling described above before the deformation modeling is applied. In particular, the segments of the image data representing the chest is extended on the posterior side using a fitted ellipsoid. These chest segments may also be smoothed using a low-pass filter to iteratively erode and dilate the chest region, and to replace points along the chest-breast tissue boundary with points at the mean positions of their nearest neighboring segments. Further, the segmentation may then be blanketed with skin (e.g., holes in the skin segmentation are filled in), an artificial table (as an elastic material) may be added to the posterior side of the model, and a layer of elastic material may be added to the posterior side of the model, to modulate the degree of deformation of tissue as it falls along the chest wall.

### B. Segmentation Coarse Graining

The number of voxels in the model is reduced and the resulting new voxels are larger, usually by a factor of 4 in each dimension. For example, voxels that are originally 1 millimeter along each edge are replaced by voxels that are 4 millimeters along each edge. In experiments, this degree of coarse-graining reduces computational cost but does not change the simulation result significantly—its error of less than about 5% was deemed adequate for the uses herein. In various embodiments, downsampling the 3D image data in this manner may be by a factor of 4 (replacing a block of 4 voxels with 1 voxel), 9, 16, etc.

### C. Translation of the Segmentation to a Finite Element Model

The elements of the finite element model can be either tetrahedral or cubic. In either case, the original voxels can be translated to these elements. If cubic elements are used, each voxel becomes a unique element. If tetrahedral elements are used, each cubic voxel is split into six unique elements. In either case, the number of vertices stays the same. Each vertex is referred to as a node. Each node has a unique ID and each element also has a unique ID. Each element can also be defined by its nodes, though each node belongs to multiple elements, and neighboring elements are coupled by sharing nodes, edges, and faces.

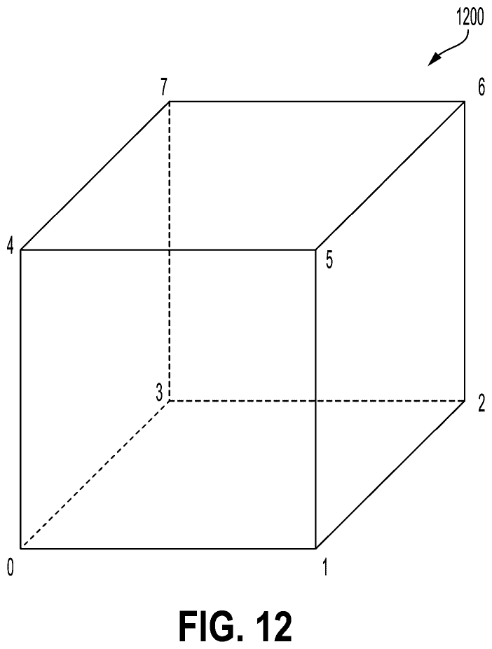

FIG. 12 depicts an example cubic voxel 1200. This voxel can be split into six tetrahedral elements respectively defined by the following sets of nodes: [0,4,6,7], [0,6,3,7], [0,2,3,6], [0,1,2,6], [0,5,1,6], and [0,4,5,6]. Other possible sets of nodes are: [0,1,3,7], [0,4,1,7], [4,5,1,7], [1,5,6,7], [1,6,2,7], and [3,1,2,7]; [0,1,2,4], [0,2,3,4], [3,7,4,2], [7,6,4,2], [2,5,6,4], and [1,5,2,4]; and [0,1,3,5], [3,4,0,5], [3,7,4,5], [5,7,6,3], [2,5,6,3], and [1,5,2,3].

Each tissue type in the segmentation is interpreted as a neo-Hookean material with its own density, Young's modulus, and Poisson ratio. Since each material (e.g., tissue types and non-tissue types) is made up of a unique group of elements, materials are therefore coupled to one another through the sharing of nodes, edges, and faces. As examples, the Young's moduli for various tissue types can be: adipose tissue—3,000 Pascals, fibroglandular tissue—10,000 Pascals, tumor—40,000 Pascals, and skin—100,000 Pascals. Densities and Poisson ratios can be free variables.

Certain sets of model components (e.g. nodes, faces, or elements) can be fixed in space and their associated degrees of freedom can be removed from the model. For example, all degrees of freedom in the chest wall and artificial posterior table may be fixed, given that it is expected that these materials are stiff compared to other material in the model. Fixing these stiffer material reduces the simulation complexity.

If using tetrahedral elements, the interface of the chest wall and breast tissue is smoothed to facilitate sliding during the simulations. This can be done in three steps.

First, some breast tissue elements adjacent to the chest wall are replaced by chest-type elements, in order to fill in the chest boundary. This results in a smoothing effect of the chest exterior.

Second, each node on the chest-breast tissue interface, and in the interior of the non-chest materials, is moved toward the mean position of its neighbor nodes. The number of iterations and amount to move along the vector connecting each node and its neighbors' center are free parameters.

Third, repeat the second step, but instead of considering all of a node's neighbors when updating its position, only consider the neighbors of the same material type. For example, only move chest nodes toward their chest-type node neighbors' center, and only move non-chest nodes toward their non-chest-type neighbors' center.

Other components of the model that do not directly use the segmentation include the force vector (defining the direction and magnitude of the force to be applied to the configuration) and several other computational parameters, such as time step size and the total simulation time over which the force will be applied.

### D. Neo-Hookean Modeling

As noted above, the tissue types are represented as Neo-Hookean materials (e.g., using a non-linear isotropic constitutive model). An unconstrained Neo-Hookean material has a non-linear stress-strain behavior, but reduces to the classical linear elasticity model for small strains and small rotations. It is derived from the following hyperelastic strain-energy density function:

$$W = \frac{μ}{2} ( {I}_{1} - 3 ) - μ ln J + \frac{λ}{2} {( ln J )}^{2} .$$

Here, I₁ is the first invariant of the right Cauchy-Green deformation tensor C and J is the determinant of the deformation gradient tensor. The relationship between the material parameters, E and v (where E is Young's modulus and v is Poisson's ratio), and the parameters used in the strain-energy function, is as follows:

$$μ = \frac{E}{2 ( 1 + ν )}$$

$$λ = \frac{ν E}{( 1 + ν ) ( 1 - 2 ν )}$$

### E. Simulating Gravitational Load on the Elements

A quasi-static simulation assumes that the system is changing slowly enough that it is always in mechanical equilibrium, meaning that forces are always balanced and there is no net acceleration (i.e., inertial terms are ignored). The equilibrium state of the system under the specified conditions can be solved. But, for numerical stability, it is done incrementally, in time steps. Although this results in a configuration at each step, one cannot interpret the resulting set of configurations as a true trajectory in a straight-forward fashion. The time in a quasi-static simulation is therefore referred to as quasi-time.

As the equilibrated supine configuration is of interest rather than the real-time trajectory of the subject moving from the prone position to the supine position, quasi-static simulations are employed. As mentioned above, for stability, the final state is still solved for in a series of steps, over which the external load increases. For example, when finding the equilibrium supine state, starting from the unloaded state, an external load on each element (a body load) of 9.8N toward the chest wall is applied. Instead of doing this directly in one step, which might cause difficulty with convergence, 9.8N is divided into a number of steps, (e.g., 10 though values of 5-20 may be used). Solutions for each of the incremental load amounts are found, up to and including 9.8N. However, the final state, in which the full 9.8N is applied, is the state of interest.

A goal of each simulation is to solve, at each time step, for the node displacements, using a set of non-linear equations that are based on material stress-strain relationships, Newton's laws, and the applied loads and boundary conditions. This may involve constructing and updating a stiffness matrix for the entire system, which stores the force-deformation relationships and coupling information for each node in the mesh. The non-linear equations are solved iteratively, through a process that involves constructing a set of linear equations that approximate the non-linear ones, solving the linear equations, plugging the results back into the original equations, determining if the convergence criteria are met, and, if not, repeating the process. To facilitate convergence, this process can allow for automatic updating of the time step size during the simulation. This results in a decrease in time step size and smaller node displacements between steps, when the solver is struggling to find a solution.

The Newton-Raphson method can be used for solving the non-linear finite element equations. This technique is more stable than quasi-Newton methods though requires re-computing the stiffness matrix during each iteration while the quasi-Newton methods do not. Nonetheless, quasi-Newton methods could be used.

### F. Simulation of Prone State to Unloaded State

The initial DCE-MRI images are captured with the subject in the prone position, meaning that the breasts are under mechanical loading due to gravity, in the posterior-to-anterior direction. Therefore, simply applying gravity in the opposite direction (anterior-to-posterior) would not mimic moving the subject from the prone state to the supine state.

Instead, a closer approximation would be to apply gravity in the posterior direction twice—once to cancel out the initial gravity vector and achieve an “unloaded” configuration, and another to simulate the force in the supine position. Although gravity is acting on the breasts in the prone position, it is not known whether there are any internal stresses present. In other words, in the prone DCE-MRI data, it is not known by how much the internal tissues are extended or compressed and in what spatial distribution. Thus, it is assumed that there are zero stresses in the model when under gravity in the prone position.

One method to find a more accurate unloaded configuration is to iteratively guess at a solution, apply gravity in the posterior-to-anterior direction to mimic going from the unloaded state to the prone state, and then compare the resulting prone state prediction to the actual DCE-MRI image data representing the breast tissue in that position. Based on the error in the predicted solution, the unloaded state is updated. This is repeated until the error is less than a pre-determined error threshold.

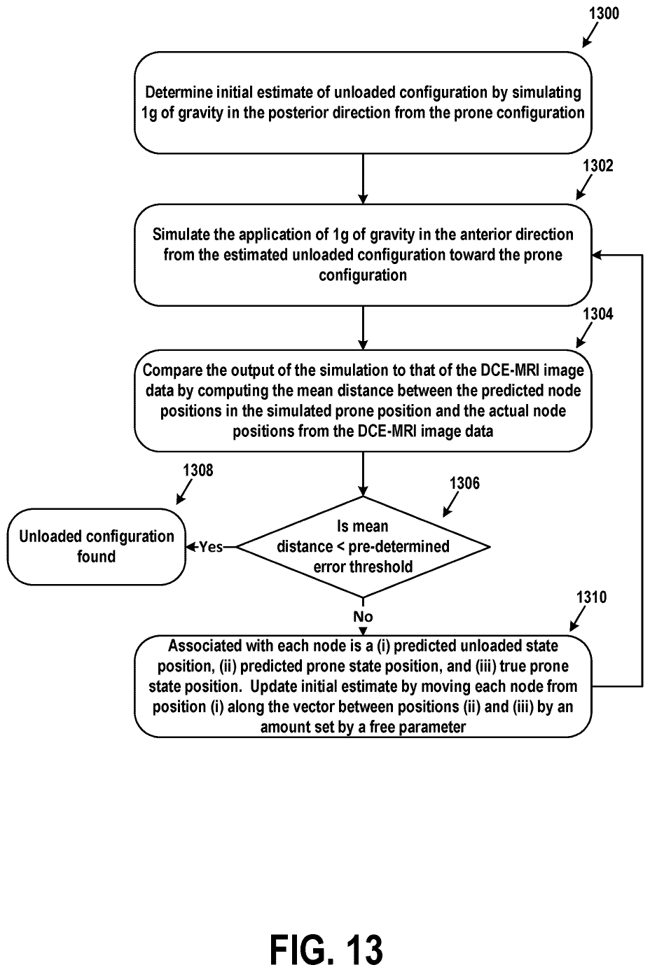

FIG. 13 depicts a procedure for carrying out these operations. Nonetheless, more or fewer steps may be present in such a procedure.

At block 1300, an initial estimate of the unloaded configuration of the breast tissue is created by simulating, e.g., using finite element analysis, the application of 1 g of gravity in the posterior direction from the prone configuration.

At block 1302, the application of 1 g of gravity in the anterior direction from the estimated unloaded configuration toward the prone configuration is simulated, e.g., using finite element analysis.

At block 1304, the output of block 1302 is compared to the true DCE-MRI image data by computing the mean distance between the predicted node positions in the simulated prone position and the actual node positions from the DCE-MRI image data.

At block 1306, it is determined whether the mean distance is less than the pre-determined error threshold. If so, the unloaded configuration has been found as depicted in block 1308.

Otherwise, at block 1310, the initial estimate is updated using the following substeps. Associated with each node is a (i) predicted unloaded state position, (ii) predicted prone state position, and (iii) true prone state position. To update the position of a node for use in a new unloaded configuration estimate, move it from position (i) along the vector between positions (ii) and (iii) by an amount set by a free parameter. This is repeated for all nodes. Then control passes back to block 1302.

This procedure continues until the solution converges and the unloaded configuration is found at block 1308.

### G. Simulation of Unloaded State to Supine State

Assuming that an estimate of the unloaded configuration has been achieved (e.g., by way of the methodology above), force due to gravity is applied in the anterior-to-posterior direction, and deformation of the breasts is simulated. To do this, interactions are added to the model that were not included when finding the unloaded state.

An influence of the deformation observed when the subject lies on her back is the sliding of breast tissue across the chest wall. This can be observed in certain cases when comparing the predicted supine breast positions to actual supine CT scans of the same subjects.

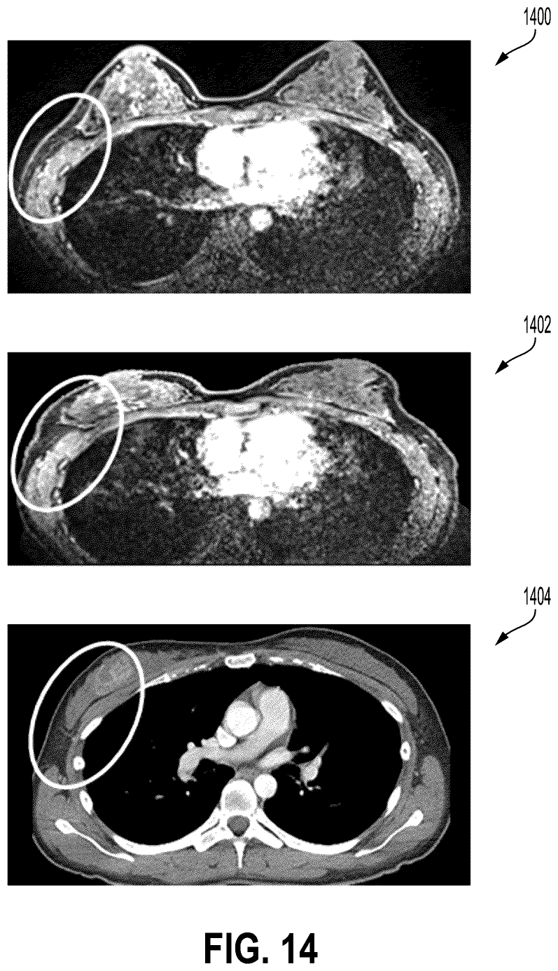

FIG. 14 provides an example. Image 1400 is a DCE-MRI taken with a subject in the prone position. Image 1402 is a predicted supine positioning without sliding interactions. Image 1404 is a supine CT scan. As shown in image 1404, the breast tissue slides somewhat laterally along the chest wall. In each image, a region of tissue, ribs, and chest has been marked with a circle. Notably, in the predicted supine MRI without sliding interactions, the breast tissue has deformed, but has not moved enough along the chest wall.

Therefore, sliding interactions along the interface between chest and other breast tissues are added to the model. This allows, for example, an adipose-type element that originally shared a face with a chest-type element to move along the chest and lose contact with its original chest-type neighbor. Notably, the model is originally constructed such that all neighboring elements share nodes and faces. To introduce sliding, elements that are neighbors are “detached” from one another across the sliding interface, adding nodes and faces to the model as a result. This ultimately results in two surfaces, made up of sets of element faces, which can slide relative to each other, along the chest wall exterior. To impose the sliding interaction, a sliding-elastic contact interaction algorithm is used, which keeps the surfaces in contact and adds friction between them. This modulates the overall amount of sliding.

The exact technique used to simulate sliding can vary. For example, it may be based on one or more of the following classes of algorithms. Sliding-node-on-facet (N2F): This technique is based on Laursen's contact formulation, which poses the contact problem as a nonlinear constrained optimization problem. The Lagrange multipliers that enforce the contact constraints are computed either using a penalty method or the augmented Lagrangian method. Sliding-facet-on-facet (F2F): This technique is similar to the N2F technique but uses Gaussian quadrature to integrate the contact equations. Doing so gives additional stability and can converges when the N2F technique does not. Sliding-elastic (SE) This sliding contact interface also uses facet-on-facet contact but differs in the linearization of the contact forces, which results in a different contact stiffness matrix compared to the previous two techniques. It may optionally be set to sustain tension to prevent contact surfaces from separating along the direction normal to the interface, while still allowing tangential sliding. This method sometimes performs better than the previous two methods for problems that are dominated by compression.

### H. Interpolation of Supine DCE-MRI Images

In order to interpolate the content of a supine DCE-MRI of breast tissue given the predicted supine node configuration and the original prone DCE-MRI data, two issues may be addressed. Each make deforming the original DCE-MRI image data into the supine configuration difficult. First, the supine node positions do not lie on a regular grid and the elements are deformed. Second, the simulation resolution is much lower than the original DCE-MRI (due to the coarse-graining described above).

Interpolation can overcome both of these issues. The array of node displacements are interpolated on a 1 millimeter-resolution grid that has the dimensions of the original DCE-MRI image. The result is an array of displacements, one for each voxel of the original DCE-MRI. Then, each DCE-MRI voxel's new location in the new, predicted image is found by adding its predicted displacement to its original location, and assigning the original voxel value to the new location.

### I. Example Operations

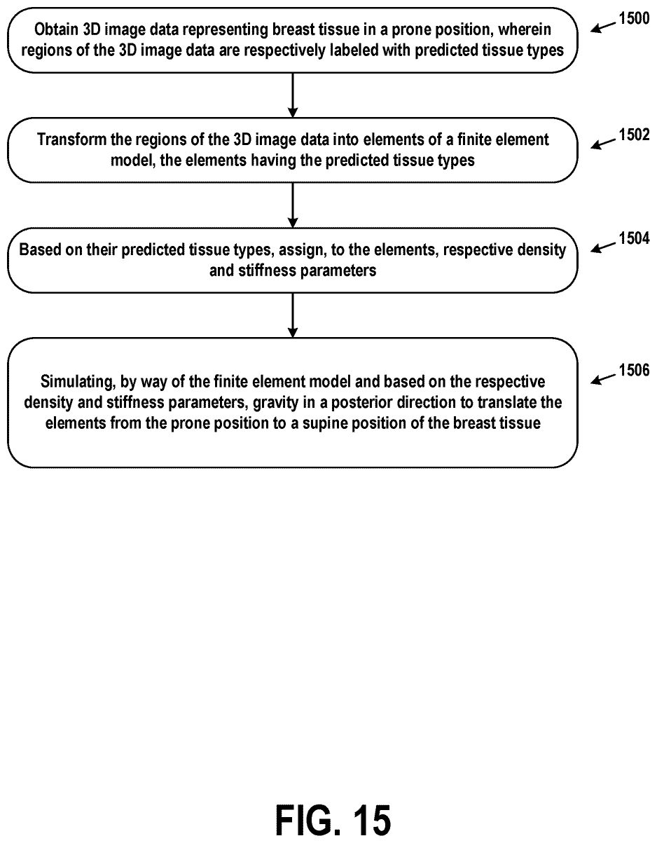

FIG. 15 is a flow chart illustrating an example embodiment. The process illustrated by FIG. 15 may be carried out by a computing device, such as computing device 100, and/or a cluster of computing devices, such as server cluster 200. However, the process can be carried out by other types of devices or device subsystems. For example, the process could be carried out by a portable computer, such as a laptop or a tablet device.

The embodiments of FIG. 15 may be simplified by the removal of any one or more of the features shown therein. Further, these embodiments may be combined with features, aspects, and/or implementations of any of the previous figures or otherwise described herein. For example, these embodiments may be combined with those of FIG. 11 in various ways.

Block 1500 may involve obtaining 3D image data representing breast tissue in a prone position, wherein regions of the 3D image data are respectively labeled with predicted tissue types. The predicted tissue types may be based on machine-learning predicted tissue labels and/or manually assigned tissue labels.

Block 1502 may involve transforming the regions of the 3D image data into elements of a finite element model, the elements having the predicted tissue types.

Block 1504 may involve, based on their predicted tissue types, assigning, to the elements, respective density and stiffness parameters.

Block 1506 may involve simulating, by way of the finite element model and based on the respective density and stiffness parameters, gravity in a posterior direction to translate the elements from the prone position to a supine position of the breast tissue.

In some implementations, simulating gravity in the posterior direction to translate the elements from the prone position to the supine position of the breast tissue comprises: simulating gravity in the posterior direction to translate the elements from the prone position to a gravity-unloaded position of the breast tissue; and simulating gravity in the posterior direction to translate the elements from the gravity-unloaded position to the supine position of the breast tissue.

In some implementations, simulating gravity to translate the elements from the gravity-unloaded position to the supine position of the breast tissue comprises laterally sliding at least some of the elements representing tumor, skin, adipose tissue, fibroglandular tissue, and vasculature across the elements representing chest wall.

In some implementations, simulating gravity to translate the elements from the prone position to the gravity-unloaded position of the breast tissue comprises repeatedly iteration of: estimating an intermediate gravity-unloaded position of the breast tissue; simulating gravity in an anterior direction from the intermediate gravity-unloaded position to a simulated prone position; determining that an iteration error value between the simulated prone position and the prone position from the 3D image data is at least a pre-determined error threshold; and updating the intermediate gravity-unloaded position by moving vertices of the elements along a vector defined by relative locations of the simulated prone position and the prone position.

Some implementations may further involve: determining that a further iteration error value is less than the pre-determined error threshold; and using the intermediate gravity-unloaded position as the gravity-unloaded position of the breast tissue.

In some implementations, simulating gravity in the posterior direction to transform the elements from the gravity-unloaded position to the supine position of the breast tissue comprises: detaching the elements representing chest wall from the elements adjacent to the chest wall representing other tissue types; modifying the elements representing the chest wall or the elements adjacent to the chest wall so that they each have respective surfaces of element faces; and applying a sliding elastic contact interaction with friction to move the elements adjacent to the chest wall into the supine position of the breast tissue.

Some implementations may further involve interpolating the elements in the supine position into further 3D image data with identified locations of the predicted tissue types.

In some implementations, the predicted tissue types include tumor, skin, adipose tissue, fibroglandular tissue, vasculature, and chest wall.

Some implementations may further involve, prior to transforming the regions of the 3D image data into the elements of the finite element model: extending the regions of the 3D image data representing chest wall on a posterior side of the breast tissue using ellipsoid fitting; smoothing the regions of the 3D image data representing the chest wall using a low-pass filter to iteratively erode and dilate a chest region; or filling in the regions of the 3D image data in which there are gaps in skin coverage of the breast tissue with representations of skin.

In some implementations, the elements of the finite element model are cubic elements.

In some implementations, the elements of the finite element model are tetrahedral elements.

In some implementations, transforming the regions of the 3D image data into elements of the finite element model comprises: replacing at least some of the elements representing breast tissue other than chest wall that are adjacent to elements representing the chest wall with elements representing the chest wall; and moving at least some vertices of the tetrahedral elements adjacent to the chest wall to a mean position of their respective neighboring vertices.

Some embodiments may further involve: prior to transforming the regions of the 3D image data into elements of the finite element model, downsampling the regions of the 3D image data by a factor of at least 4. However, a factor of 2 or other factors may be used.

Some embodiments may involve interpolating the elements in the supine position into further 3D image data with identified locations of the predicted tissue types by interpolating displacements of vertices of the elements in the supine position from the prone position on a grid of locations without downsampling, and moving the regions of the 3D image data by their respective displacements.

### VII. Example Technical Improvements

These embodiments provide technical solutions to technical problems. One technical problem being solved is accurate multi-tissue segmentation of breast tissue into tissue types from 3D image data. In practice, this is problematic because the locations of these tissue types can be critical to consider during breast cancer surgery planning as well as the actual surgery. Another technical problem being solved is determining how these locations move when a subject is shifted from the prone position (laying face-down) to the supine position (laying face-up).

In the prior art, these problems could not be solved with high enough accuracy to be practically reliable. For instance, the prior art employs prediction networks that often fail to identify tumors present in the prone image data. The prior art also cannot accurately model the impact of gravity on various tissue types as well as interactions between these tissue types under gravitational pull. Thus, prior art techniques often rely on subjective decisions and experiences of radiologists, surgeons, and other medical professionals, which can lead to wildly varying outcomes from instance to instance. Thus, these techniques did little if anything to address providing clinically accurate localizations of breast tumors that are usable for surgical procedures.

The embodiments herein overcome these limitations by using several machine learning techniques to segment and identify types of breast tissue—including tumors—with high accuracy. In addition to synthesizing these machine learning techniques in a manner that increases accuracy, post-processing steps can take anatomical knowledge into account to further improve the results. Moreover, these embodiments adapt finite element modeling to account for changes in tissue locations as the subject is moved between positions. Such adaptations include lateral movements of breast tissue that cannot be representation by traditional models. In this manner, surgical planning can be accomplished in a more accurate and robust fashion.

Other technical improvements may also flow from these embodiments, and other technical problems may be solved. Thus, this statement of technical improvements is not limiting and instead constitutes examples of advantages that can be realized from the embodiments.

### VIII. Closing

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as non-transitory computer readable media that store data for short periods of time like register memory and processor cache. The non-transitory computer readable media can further include non-transitory computer readable media that store program code and/or data for longer periods of time. Thus, the non-transitory computer readable media may include secondary or persistent long-term storage, like ROM, optical or magnetic disks, solid-state drives, or compact disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

1. A computer-implemented method comprising:
obtaining 3D image data of breast tissue;
applying a tumor localization procedure to the 3D image data, wherein applying the tumor localization procedure includes using a tumor localization neural network ensemble to predict 3D bounding boxes of tumor locations within the 3D image data;
applying a tumor segmentation procedure to the 3D image data, wherein applying the tumor segmentation procedure includes using a tumor segmentation neural network to predict first probabilities that a first set of locations within the breast tissue contain tumor tissue;
applying a multi-tissue segmentation procedure to the 3D image data, wherein applying the multi-tissue segmentation procedure includes using a tissue segmentation neural network ensemble to predict second probabilities that a second set of locations within the breast tissue contain one or more types of non-tumor tissue, and combining the second probabilities using a weighted average; and
determining most likely tissue types for regions of the 3D image data based on the 3D bounding boxes, the first probabilities from the tumor segmentation procedure, the second probabilities as combined from the multi-tissue segmentation procedure, and anatomical feasibilities of the breast tissue.

2. The computer-implemented method of claim 1, wherein using the tumor localization neural network ensemble to predict the 3D bounding boxes of tumor locations within the 3D image data comprises:
flattening the 3D image data into 2D image data for two planes of the 3D image data;
using the tumor localization neural network ensemble to predict 2D tumor locations within the 2D image data; and
merging the 2D tumor locations into the 3D bounding boxes.

3. The computer-implemented method of claim 2, wherein merging the 2D tumor locations into 3D bounding boxes comprises determining intersections of the 2D tumor locations across the two planes.

4. The computer-implemented method of claim 2, wherein determining most likely tissue types for regions of the 3D image data comprises one or more of: eliminating or replacing predicted most likely tissue types of tumor located outside of the 3D bounding boxes, or eliminating or replacing anatomically impossible predicted most likely tissue types.

5. The computer-implemented method of claim 2, wherein the two planes of the 3D image data are selected from axial, sagittal, or coronal planes.

6. The computer-implemented method of claim 2, wherein the tumor localization neural network ensemble comprises two neural networks that were respectively trained on labeled maximum intensity projections of tumor locations within the two planes.

7. The computer-implemented method of claim 1, wherein applying the tumor segmentation procedure comprises dividing the 3D image data into 3D windows, and wherein the first set of locations comprises the 3D windows.

8. The computer-implemented method of claim 1, wherein applying the multi-tissue segmentation procedure comprises dividing the 3D image data into further 3D windows, and wherein the second set of locations comprises the further 3D windows.

9. The computer-implemented method of claim 1, wherein the 3D image data comprises two or more DCE-MRI images from different post-contrast injection time points or from two or more different MRI modalities.

10. The computer-implemented method of claim 9, further comprising:
aligning the two or more DCE-MRI images;
modifying the two or more DCE-MRI images to equalize pixel spacing in each dimension; and
standardizing intensities of pixels within the two or more DCE-MRI images.

11. The computer-implemented method of claim 1, wherein the tumor segmentation neural network was trained on randomly-selected labeled locations within 3D training images of breast tissue, wherein the randomly-selected labeled locations are biased toward tumor locations over non-tumor locations.

12. The computer-implemented method of claim 1, wherein the tissue segmentation neural network ensemble comprises a plurality of tissue segmentation neural networks, one for each of a plurality of non-tumor tissue types, and wherein each of the plurality of tissue segmentation neural networks was trained on labeled locations within 3D training images of their respective non-tumor tissue types.

13. The computer-implemented method of claim 12, wherein the plurality of tissue segmentation neural networks include one for each of skin, adipose tissue, fibroglandular tissue, vasculature, and chest wall.

14. The computer-implemented method of claim 1, wherein tissue segmentation neural network ensemble also predicts probabilities that each of the first set of locations contain air, the method further comprising:
determining a nipple location on a breast represented in 3D image data based on a confluence of physically adjacent or overlapping predictions of air, glandular tissue, and skin within the 3D image data.

15. The computer-implemented method of claim 1, wherein the 3D image data represents the breast tissue in a prone position, the computer-implemented method further comprising:
transforming the regions of the 3D image data into elements of a finite element model, the elements having their respective most likely tissue types;
based on their respective most likely tissue types, assigning, to the elements, respective density and stiffness parameters;
simulating, by way of the finite element model and based on the respective density and stiffness parameters, gravity in a posterior direction to translate the elements from the prone position to a gravity-unloaded position of the breast tissue;
simulating, by way of the finite element model, gravity in the posterior direction to translate the elements from the gravity-unloaded position to a supine position of the breast tissue; and
interpolating the elements in the supine position into further 3D image data with identified locations of the respective most likely tissue types.

16. A non-transitory computer-readable medium, having stored thereon program instructions that, upon execution by a computing system, cause the computing system to perform operations comprising:
obtaining 3D image data of breast tissue;
applying a tumor localization procedure to the 3D image data, wherein applying the tumor localization procedure includes using a tumor localization neural network ensemble to predict 3D bounding boxes of tumor locations within the 3D image data;
applying a tumor segmentation procedure to the 3D image data, wherein applying the tumor segmentation procedure includes using a tumor segmentation neural network to predict first probabilities that a first set of locations within the breast tissue contain tumor tissue;
applying a multi-tissue segmentation procedure to the 3D image data, wherein applying the multi-tissue segmentation procedure includes using a tissue segmentation neural network ensemble to predict second probabilities that a second set of locations within the breast tissue contain one or more types of non-tumor tissue, and combining the second probabilities using a weighted average; and
determining most likely tissue types for regions of the 3D image data based on the 3D bounding boxes, the first probabilities from the tumor segmentation procedure, the second probabilities as combined from the multi-tissue segmentation procedure, and anatomical feasibilities of the breast tissue.

17. A system comprising:
one or more processors; and
memory, containing program instructions that, upon execution by the one or more processors, cause the system to perform operations comprising:
obtaining 3D image data of breast tissue;
applying a tumor localization procedure to the 3D image data, wherein applying the tumor localization procedure includes using a tumor localization neural network ensemble to predict 3D bounding boxes of tumor locations within the 3D image data;
applying a tumor segmentation procedure to the 3D image data, wherein applying the tumor segmentation procedure includes using a tumor segmentation neural network to predict first probabilities that a first set of locations within the breast tissue contain tumor tissue;
applying a multi-tissue segmentation procedure to the 3D image data, wherein applying the multi-tissue segmentation procedure includes using a tissue segmentation neural network ensemble to predict second probabilities that a second set of locations within the breast tissue contain one or more types of non-tumor tissue, and combining the second probabilities using a weighted average; and
determining most likely tissue types for regions of the 3D image data based on the 3D bounding boxes, the first probabilities from the tumor segmentation procedure, the second probabilities as combined from the multi-tissue segmentation procedure, and anatomical feasibilities of the breast tissue.

18-33. (canceled)

34. The non-transitory computer-readable medium of claim 16, wherein using the tumor localization neural network ensemble to predict the 3D bounding boxes of tumor locations within the 3D image data comprises:
flattening the 3D image data into 2D image data for two planes of the 3D image data;
using the tumor localization neural network ensemble to predict 2D tumor locations within the 2D image data; and
merging the 2D tumor locations into the 3D bounding boxes.

35. The non-transitory computer-readable medium of claim 34, wherein determining most likely tissue types for regions of the 3D image data comprises one or more of: eliminating or replacing predicted most likely tissue types of tumor located outside of the 3D bounding boxes, or eliminating or replacing anatomically impossible predicted most likely tissue types.

36. The non-transitory computer-readable medium of claim 16, wherein the 3D image data represents the breast tissue in a prone position, the operations further comprising:
transforming the regions of the 3D image data into elements of a finite element model, the elements having their respective most likely tissue types;
based on their respective most likely tissue types, assigning, to the elements, respective density and stiffness parameters;
simulating, by way of the finite element model and based on the respective density and stiffness parameters, gravity in a posterior direction to translate the elements from the prone position to a gravity-unloaded position of the breast tissue;
simulating, by way of the finite element model, gravity in the posterior direction to translate the elements from the gravity-unloaded position to a supine position of the breast tissue; and
interpolating the elements in the supine position into further 3D image data with identified locations of the respective most likely tissue types.
